SEQUENCE LISTING (3 sequences)

SEQ ID NO: 1 - DNA, 7 bp, Homo sapiens
acgactg

SEQ ID NO: 2 - DNA, 7 bp, Homo sapiens
gcactat

SEQ ID NO: 3 - DNA, 7 bp, Homo sapiens
aggcttg

(19) United States
(12) Patent Application Publication
(10) Pub. No.: US 2011/0014625 A1
(43) Pub. Date: Jan. 20, 2011

(54) System and Method for Determining the Health of a Subject Using Polymorphic Risk Markers

(75) Inventors: Steven A. Belinsky, Albuquerque, NM (US); Shuguang Leng, Albuquerque, NM (US)

Correspondence Address: PEACOCK MYERS, P.C., 201 THIRD STREET, N.W., SUITE 1340, ALBUQUERQUE, NM, 87102, US

(73) Assignee: LOVELACE RESPIRATORY RESEARCH INSTITUTE, Albuquerque, NM (US)

(21) Appl. No.: 12/922,654

(22) PCT Filed: Mar. 16, 2009
(86) PCT No.: PCT/US09/37248

§ 371 Date: Sep. 14, 2010

Related U.S. Application Data
(60) Provisional application No. 61/037,052, filed on Mar. 17, 2008.

(51) Int. Cl. C12Q 1/68

(52) U.S. Cl. 435/6

(57) ABSTRACT

A system and method for predicting the health of a subject comprising obtaining nucleic acid sequence data about the subject. Identifying at least one polymorphic risk marker associated with a change in promoter methylation of a gene associated with lung cancer; and predicting the health of the subject from a presence of at least one polymorphic risk marker identified and kits associated therewith.

### CROSS-REFERENCE TO RELATED APPLICATIONS

This application claims priority to and the benefit of the filing of U.S. Provisional Patent Application Ser. No. 61/037,052, entitled SYSTEM AND METHOD FOR DETERMINING THE HEALTH OF A SUBJECT USING DNA DOUBLE STAND BREAK REPAIR AND GENE METHYLATION POLYMORPHIC RISK MARKERS, filed on Mar. 17, 2008 and the specification and claims thereof are incorporated herein by reference.

### INCORPORATION BY REFERENCE OF MATERIAL SUBMITTED IN A TXT FILE

A sequence listing titled SNP.ST25.txt created on Mar. 13, 2009 having 792 Bytes and is ASCII compliant is filed herewith to satisfy 37 CFR 1.821(c). The information recorded in the electronic form is identical to the sequence listing in the application.

### BACKGROUND OF THE INVENTION

Gene promoter hypermethylation in sputum is a biomarker for predicting lung cancer. Identifying factors that predispose smokers to methylation of multiple gene promoters in the lung could impact strategies for early detection and chemoprevention.

Lung cancer, the leading cause of cancer mortality in both men and women in the United States, now accounts for approximately 30% of all deaths from cancer. The 5-year survival rate of lung cancer patients is about 14%. The discovery of field cancerization in the respiratory tract of smokers prompted studies leading to the discovery that inactivation of genes such as p16 bp promoter hypermethylation occurs in precursor lesions to non-small cell lung cancer. This finding suggested that methylation, when detected in exfoliated cells within sputum, could serve as a biomarker for the early stages of lung carcinogenesis.

The precise mechanisms by which carcinogens disrupt the cells' capacity to maintain the normal epigenetic code during DNA replication and repair are largely unknown. Smoking accounts for >90% of lung cancer. Carcinogens within tobacco induce single- and double-strand breaks (DSBs) in DNA. Reduced capacity for repair of DNA damage has been associated with lung cancer. DNA damage, manifested through DSBs, could in part be responsible for the acquisition of aberrant gene promoter methylation during lung carcinogenesis. For example, the prevalence of promoter methylation of the p16 gene is significantly greater in adenocarcinomas from workers occupationally exposed to plutonium, an exposure that predominantly produces DSBs, than in cancer from unexposed smokers. The prevalence of p16 methylation increased with increasing plutonium exposure. In a second study, the prevalence of methylation of the estrogen receptor-α gene promoter was greater in plutonium-induced adenocarcinomas in rodent lung tumors compared to tumors induced by NNK [4-(methylnitrosamino)-1-(3-pyridyl)-1-butanone], diesel exhaust, or carbon black exposures which mainly induce single-strand breaks of DNA (Carcinogensis 2005; 26:1481-7).

### BRIEF DESCRIPTION OF THE FIGURES

FIG. 1 illustrates (panel A) chromatid breaks in control and cases in and (panel B) association with gene promoter DNA repair capacity associated with gene promoter methylation in sputum and (Panel C) an ROC curve.

FIG. 2 illustrates (Panel A) SNP in repair genes is associated with gene promoter methylation in sputum and promoter activity in MRE11A and (Panel B) SNP in repair genes is associated with gene promoter methylation in sputum and promoter activity in MRE11A with 10 or 5 SNPs considered.

### BRIEF DESCRIPTION OF THE EMBODIMENTS OF THE INVENTION

According to one embodiment of the present invention, the health of a subject is predicted by the method comprising obtaining nucleic acid sequence data about the subject. At least one polymorphic risk marker is identified which is associated with a change in promoter methylation of a gene associated with lung cancer. For example he subject is a human. The health of the subject is predicted from a presence of at least one polymorphic risk marker identified. In a preferred embodiment obtaining a nucleic acid sequence data is obtained for one or more of the flowing genes XRCC3, DNA-PKc, NBN, LIG4, XRCC2, CHEK1, MRE11A, CHEK2, RAD50, and KU80. In a more preferred embodiment the at least one polymorphic risk marker is selected from the group consisting of: an allele A in marker rs537046 of gene CHEK1; an allele C in marker rs5762763 of gene CHEK2; an allele C in marker rs1151402 of gene LIG4; an allele T in marker rs7117042 of gene MRE11A; an allele T in marker rs6998169 of gene NBN; an allele A in marker rs7830743 of gene DNA-PKc; an allele G in marker rs2244012 of gene RAD50; an allele C in marker rs3218400 of gene XRCC2; an allele C in marker rs2295146 of gene XRCC3; and an allele A in marker rs828911 of gene KU80. In another preferred embodiment determining a risk includes identifying the presence of a five polymorphic risk markers selected from the group consisting of: an allele C in marker rs5762763 of gene CHEK2; an allele T in marker rs7117042 of gene MRE11A; an allele T in marker rs6998169 of gene NBN; an allele A in marker rs7830743 of gene DNA-PKc; and an allele C in marker rs2295146 of gene XRCC3. In another preferred embodiment a gene associated with cancer is selected from the group consisting of p16, MGMT, DAPK, RASSF1A, PAX5-α, PAX5-β, GATA4, and GATA5. In another preferred embodiment, determining the health of a subject comprises comparing the obtained nucleic acid sequence data to a database containing correlation data between polymorphic risk markers and risk factors to provide a score relating to the health of the subject. For example the presence of the five polymorphic risk markers from the group are present in 7 or more of 10 possible alleles predicts the health of the subject. In a more preferred embodiment detecting a polymorphic risk marker that is in linkage disequilibrium with one or more of the at least one polymorphic risk markers identified in claim 4. For example the polymorphic risk markers in linkage disequilibrium with a polymorphic risk marker are selected from table 7. For example, linkage disequilibrium is defined by numerical values of r.̂2 of at least 0.8.

In another embodiment in a nucleic acid sample of the subject a polymorphic risk marker is detected for one or more of the genes selected from the group consisting of XRCC3, DNA-PKc, NBN, LIG4, XRCC2, CHEK1, MRE11A, CHEK2, RAD50, and KU80. For example the nucleic acid sample comprises DNA, RNA or both. The nucleic acid sample is amplified for example by a polymerase chain reaction. During amplification the polymorphic risk marker is detected by amplification such as a polymerase chain reaction or sequencing.

In another embodiment, a kit for detecting a polymorphic risk marker associated with a change in promoter methylation of a gene comprises reagents for selectively detecting at least one allele of at least one polymorphic risk marker from XRCC3, DNA-PKc, NBN, LIG4, XRCC2, CHEK1, MRE11A, CHEK2, RAD50, and KU80 in the genome of an individual, wherein the polymorphic risk marker is selected from the group consisting of the polymorphic risk markers listed in Table 7, and markers in linkage disequilibrium therewith.

In yet another embodiment, a computer-readable medium having computer executable instructions for predicting the health of a subject at risk for developing lung cancer the computer readable medium comprising data indicative of at least one polymorphic risk marker from each gene selected from the group consisting of XRCC3, DNA-PKc, NBN, LIG4, XRCC2, CHEK1, MRE11A, CHEK2, RAD50, and KU80 and a routine stored on the computer readable medium and adapted to be executed by a processor to predict the health of a subject at risk for developing lung cancer when one or more from the at least one polymorphic risk marker from each gene selected from the group consisting of XRCC3, DNA-PKc, NBN, LIG4, XRCC2, CHEK1, MRE11A, CHEK2, RAD50, and KU80 is present in a nucleic acid sequence data obtained from a subject.

In yet another embodiment, a method of aiding in a diagnosis of a subject suspected of lung cancer, the method comprising the steps of obtaining nucleic acid sequence data about the subject. The presence of one or more polymorphic risk markers from the nucleic acid sequence data is identified. The number of polymorphic risk markers is compared to a look up table. A score is assigned based upon the number of polymorphic risk markers present. Based on one or more data points such as the score, subject health information, and/or predisposition, whether the subject has a risk of lung cancer is determined. The health of the subject is determined. In a preferred embodiment, the one or more polymorphic risk markers are selected from the group consisting of an allele A in marker rs537046 of gene CHEK1; an allele C in marker rs5762763 of gene CHEK2; an allele C in marker rs1151402 of gene LIG4; an allele T in marker rs7117042 of gene MRE11A; an allele T in marker rs6998169 of gene NBN; an allele A in marker rs7830743 of gene DNA-PKc; an allele G in marker rs2244012 of gene RAD50; an allele C in marker rs3218400 of gene XRCC2; an allele C in marker rs2295146 of gene XRCC3; and an allele A in marker rs828911 of gene KU80. The method may further include obtaining at least one biometric parameter from the subject. For example information which may be obtained from a health survey conducted by a health care provider.

In a preferred embodiment, the at least one biometric parameter includes the smoking history of the subject. In a preferred embodiment one or more of the methods disclosed herein is a computer implemented method. For example a computer implemented method for aiding in a diagnosis of a subject suspected of lung cancer.

Another aspect of the present invention provides a method of predicting the likelihood that a subject will develop lung cancer.

Yet another method of the present invention provides for identifying a subject at risk for developing lung cancer. Yet another aspect of the present invention includes diagnosis, prognosis, or monitoring a subject with the system and method disclosed herein.

Another aspect of the present invention provides for a method for evaluating a subject who has a predisposition for developing lung cancer should receive further testing

Another aspect of the present invention is a method of determining a subjects likelihood of longevity.

One aspect of the present invention provides an in vivo association between DRC and gene promoter methylation, both through a functional assay and genetic variants in genes within the double-strand break repair pathway.

Another aspect of the present invention is the identification of an activity deficit of the MRE11A gene that plays a critical role in recognition of double-strand break DNA damage and activation of the ATM gene. The mechanism underlying this association could in part be mediated by the genes that are recruited to sites of DSBs, and the resultant modification of chromatin to facilitate repair.

One aspect of the present invention provides for identification of double-strand break repair capacity (DSBRC) and specific genes within this pathway as a critical determinant for gene promoter hypermethylation.

One aspect of the present invention provides for validation of the polymorphisms as an indicator of the health of the subject and/or methylation index. Genetic variants associated with promoter hypermethylation could be used to identify young smokers who would be most susceptible to induction of preneoplasia, and thus, should receive chemoprevention. In addition, the integration of these genetic variants with detection of gene promoter hypermethylation in sputum in long-term heavy smokers will provide a diagnostic test for incident lung cancer and impact long-term survival from this fatal disease.

### DETAILED DESCRIPTION OF THE INVENTION

As used herein “a” means one or more unless otherwise defined.

As used herein, an “allele” refers to the nucleotide sequence of a given locus (position) on a chromosome. Genomic DNA from an individual contains two alleles for any given polymorphic marker, representative of each copy of the marker on each chromosome.

A “haplotype,” as described herein, refers to a segment of genomic DNA within one strand of DNA that is characterized by a specific combination of alleles arranged along the segment. For diploid organisms such as humans, a haplotype comprises one member of the pair of alleles for each polymorphic marker or locus.

The nucleotide sequence of a gene, as used herein, encompasses coding regions, referred to as exons, intervening, non-coding regions, referred to as introns, and upstream or downstream regions. Upstream or downstream regions can include regions of the gene that are transcribed but not part of an intron or exon, or regions of the gene that comprise, for example, binding sites for factors that modulate gene transcription.

The genomic sequence for the CHEKI gene is included in GenBank accession number NM001274.

The genomic sequence for the CHEK2 gene is included in GenBank accession number NM00100573, NM007194, NM145862.

The genomic sequence for the LIG4 gene is included in GenBank accession number NM002312, NM206937.

The genomic sequence for the MRE11 gene is included in GenBank accession number NM005590, NM005591.

The genomic sequence for the NMB gene is included in GenBank accession number NM001024688, NM002485.

The genomic sequence for the DNA-PKC gene is included in GenBank accession number NM006904.

The genomic sequence for the RAD50 gene is included in GenBank accession number NM005732, NM133482.

The genomic sequence for the XRCC2 gene is included in GenBank accession number NM005431.

The genomic sequence for the XR CC3 gene is included in GenBank accession number is NM005432.

The genomic sequence for the KU80 gene is included in GenBank accession number is NM021141.

As used herein “Linkage Disequilibrium” (“LD”) refers to alleles at different loci that are not associated at random. If the alleles are in positive linkage disequilibrium, then the alleles occur together more often than expected assuming statistical independence. Conversely, if the alleles are in negative linkage disequilibrium, then the alleles occur together less often than expected assuming statistical independence.

As used herein “Odds Ratio” (“OR”) refers to the ratio of the odds of the disease for individuals with the marker (allele or polymorphism) relative to the odds of the disease in individuals without the marker (allele or polymorphism).

As used herein “Single Nucleotide Polymorphism (SNP)” means a DNA sequence variation occurring when a single nucleotide—Adenine=A, Thymine=T, Cytosine=C, or Guanine=G—at a specific location in the genome differs between members of a species or between paired chromosomes in an individual. Most SNP polymorphisms have two alleles. Each individual is in this instance either homozygous for one allele of the polymorphism (i.e. both chromosomal copies of the individual have the same nucleotide at the SNP location), or the individual is heterozygous (i.e. the two sister chromosomes of the individual contain different nucleotides). The SNP nomenclature as reported herein refers to the official Reference SNP (rs) ID identification tag as assigned to each unique SNP by the dbSNP database at the National Center for Biotechnological Information (NCBI) as of Mar. 6, 2009.

Alleles for SNP markers as referred to herein refer to the bases A, C, G or T as they occur at the polymorphic site in the SNP assay employed. The person skilled in the art will however realize that by assaying or reading the opposite DNA strand, the complementary allele can in each case be measured. Thus, for a polymorphic site (polymorphic marker) characterized by an A/G polymorphism, the assay employed may be designed to specifically detect the presence of one or both of the two bases possible, i.e. A and G. Alternatively, by designing an assay that is designed to detect the opposite strand on the DNA template, the presence of the complementary bases T and C can be measured. Quantitatively (for example, in terms of relative risk), identical results would be obtained from measurement of either DNA strand (+strand or −strand).

A “polymorphic risk marker”, sometimes referred to as a “marker”, as described herein, refers to a genomic polymorphic site identified by rs number. Each polymorphic risk marker has at least two sequence variations characteristic of particular alleles at the polymorphic site (major allele and minor allele). Thus, genetic association to a polymorphic risk marker implies that there is association to at least one specific allele of that particular polymorphic risk marker. The marker can comprise any allele of any variant type found in the genome, including single nucleotide polymorphisms (SNPs). Polymorphic risk markers can be of any measurable frequency in the population. The major or the minor allele can be the polymorphic risk marker.

A “nucleic acid sample” is a sample obtained from an individual that contains nucleic acid (DNA or RNA). In certain embodiments, i.e. the detection of specific polymorphic risk markers and/or haplotypes, the nucleic acid sample comprises genomic DNA. Such a nucleic acid sample can be obtained from any source that contains genomic DNA, including as a blood sample, sample of amniotic fluid, sample of cerebrospinal fluid, or tissue sample from skin, muscle, buccal or conjunctival mucosa (buccal swab), placenta, gastrointestinal tract or other organs.

A “variant”, as described herein, refers to a segment of DNA that differs from the reference DNA. A “marker” or a “polymorphic risk marker”, as defined herein, is a variant. Alleles that differ from the reference are referred to as “variant” alleles.

A “computer-readable medium”, is an information storage medium that can be accessed by a computer using a commercially available or custom-made interface. Exemplary computer-readable media include memory (e.g., RAM, ROM, flash memory, etc.), optical storage media (e.g., CD-ROM), magnetic storage media (e.g., computer hard drives, floppy disks, etc.), punch cards, or other commercially available media. Information may be transferred between a system of interest and a medium, between computers, or between computers and the computer-readable medium for storage or access of stored information. Such transmission can be electrical, or by other available methods, such as IR links, wireless connections, etc.

D′=1 means there is no recombination between these two SNPs. R̂² further considers the different allele frequencies between any two given SNPs. R̂²=1 means perfect linkage disequilibrium (no recombination and allele frequencies for the two SNPs are the same). From a SNP list, we can tell that the R² for some SNP pairs whose D′ is equal to one may be less than one.

A large panel of genes was examined for their ability to predict lung cancer in a nested case-control study. According to one embodiment of the present invention, a combination of six genes was identified whose methylation in sputum predicted lung cancer prior to clinical diagnosis with both a sensitivity and specificity of 65%. According to another embodiment of the present invention one or more of the six genes were identified whose methylation in sputum predicted lung cancer prior to clinical diagnosis with both a sensitivity and specificity to be significant.

One embodiment of the present invention provides a system or method that identifies high methylation index and correlates this index with a reduced capacity to repair DSBs in a human subject. This information is useful to predict the health of the subject. In addition, sequence variation in genes from the DSB repair pathway is identified that predict for high methylation index.

One aspect of the present invention provides that double-strand break repair capacity and sequence variation in genes in this pathway are associated with a high methylation index in a cohort of current and former cancer-free smokers.

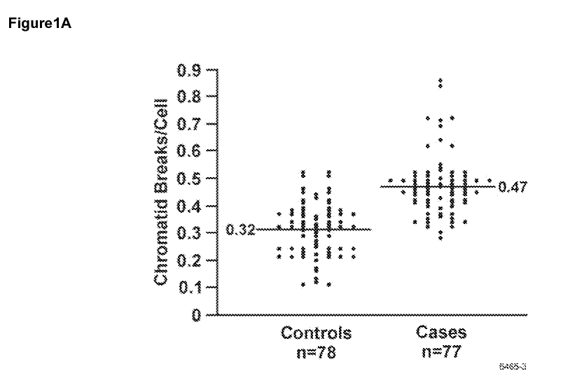

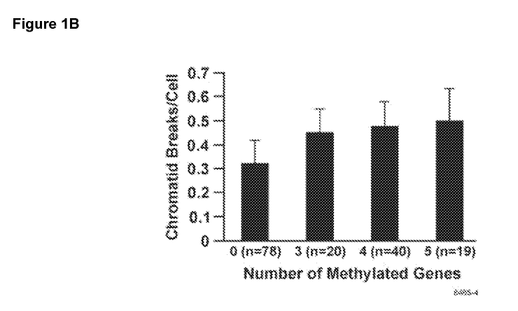

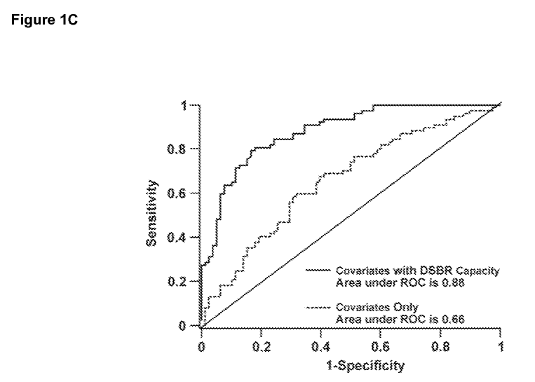

Referring now to FIG. 1, a graph illustrates that DNA repair capacity is associated with gene promoter methylation in sputum. FIG. 1, panel (A) shows that Bleomycin treatment causes an increased number of chromatid breaks/cell in lymphocytes from cases (methylated group) having an n=77 showing breaks/cell of about 0.47 as compared to controls (unmethylated group; p<0.0001) having an n=78 with breaks/cell of about 0.32. FIG. 1, panel (B) illustrates positive association between number of methylated genes and chromatid breaks/cell. Sample size for each group is indicated in parentheses. FIG. 1, panel (C) shows receiver operator curve (ROC) curve comparing sensitivity and specificity of DNA repair capacity for classifying cases and controls. The covariates included in the ROC curve were age at sputum collection, sex, race, current smoking status, and pack years. The broken line illustrates covariates only area under ROC is 0.66. The solid line illustrates covariates with DSBRC area under ROC is 0.88.

A 50% reduction in the mean level of double-strand break repair capacity was seen in lymphocytes from smokers with a high methylation index, defined as ≧3 of 8 genes selected from (p 16, MGMT, PAX5-α, PAX5-β, GATA4, GATA5, DAPK, RASSF1A) methylated in sputum, compared to smokers with no genes methylated. The classification accuracy for predicting risk for methylation was 88%. SNPs within the MRE11A, CHEK2, XRCC3, DNA-Pkc, and NBN DNA repair genes were highly associated with the methylation index and the health of a subject. A 14.5-fold increased odds for high methylation was seen for persons with ≧7 risk alleles out of a possible 10 alleles of these genes. Promoter activity of the MRE11A gene that plays a critical role in recognition of DNA damage and activation of Ataxia Telanqiectasia Mutated (ATM) was reduced in persons with the risk allele. This is the first population-based study to identify double-strand break DNA repair capacity and specific genes within this pathway as critical determinants for gene methylation in sputum, that is, in turn, associated with elevated risk for lung cancer and/or the health of a subject.

High Methylation Index as used herein is defined as the methylation of three or more gene-specific promoters selected from p16, MGMT, PAX5-α, PAX5-β, GATA4, GATA5, DAPK, RASSF1A detected in sputum.

Gene Methylation in Sputum. Gene promoter methylation was assessed in sputum from 824 members of the cohort, a cohort of current and former cancer-free smokers (Table 1). Methylation of an eight-gene panel that included p16, O⁶-methylguanine-DNA methyltransferase (MGMT), death associated protein kinase (DAPK), ras effector homolog 1 (RASSF1A), GATA4, GATA5, PAX5-α, and PAX5-β was evaluated. Methylation of these genes has been associated with increased risk for lung cancer (Cancer Res. 2006; 66: 3338-44). The prevalence of methylation ranged from 1.2% for RASSF1A to 31% for GATA4 and was not associated with family history for lung cancer (Table 5). Nineteen percent of cohort members were methylated for three or more genes (Table 5). Our previous nested case-control study within a Cohort revealed that methylation of ≧3 genes from a 6-gene panel (excluding GATA4 and PAX5-α) was associated with a 6.5-fold increased risk for lung cancer.

Repair capacity associates with methylation index. The mutagen sensitivity assay was used to assess double-strand break repair capacity (DSBRC) (Int. J. Cancer 1989; 43: 403-9). The mutagen sensitivity assay as used herein is a quantitative measurement of breaks within the 46 chromosomes of a cell following exposure to bleomycin, a radiomimetic agent that induces double-strand breaks in DNA. The greater the number of breaks, the worse the DNA repair capacity. Thus, the number of chromatid breaks induced in lymphocytes following exposure to bleomycin was used to measure DSBRC. We selected persons from our cohort who exhibited a high (cases [≧3 methylated genes]) or low (controls [zero of eight genes methylated]) methylation index because of the increased risk for lung cancer seen in nested, case-control study when 3 or more genes were methylated in sputum. Cryopreserved lymphocytes were available for assessment of DSBRC for 77 cases and 78 controls. Demographics and smoking history for cases and controls are detailed in Table 1. A highly statistically significant difference was seen in DSBRC (p<0.001) between cases and controls with a mean number of chromosome breaks per cell of 0.47±0.11 and 0.32±0.10, respectively (FIG. 1A). The mean number of bleomycin-induced chromatid breaks per cell was significantly higher in cases than in controls when subjects were stratified by age, sex, race, chronic airway obstruction, pack years, and smoking status indicating that none of these covariates were major confounders for the strong association seen between DSBRC and methylation index (Table 6).

We further classified the cases into three groups based on the number of methylated genes (3, 4 and ≧5 methylated genes) and found that the number of chromatid breaks per cell induced by bleomycin increased with the increasing number of methylated genes in sputum (p<0.001; FIG. 1B). Age did not differ in cases with 3, 4 and ≧5 methylated genes. Finally, after adjusting for sex, race, current smoking status, cigarette pack years, seeding number of lymphocytes, cryopreservation time, and log-transformed spontaneous chromatid breaks per cell, age was the only factor significantly associated with chromatid breaks induced by bleomycin in both cases and controls (Table 6). The reduction of DNA repair capacity with age is well established and supports the accuracy of the mutagen sensitivity assay in this study.

A receiver operator characteristic (ROC) curve was generated to determine how well DSBRC distinguished cases from controls. The ROC curve demonstrates that DSBRC significantly (p<0.0001) increased the classification accuracy from 66% to 88% for predicting risk for promoter methylation (FIG. 1C). With the sensitivity set at 80%, the false positive rate was <20%.

SNPs within DNA repair genes and risk for methylation. DNA repair capacity strongly predicts for high methylation index, and has high heritability. It was unexpectedly observed that variants in genes involved in repair were predictive of a change in promoter methylation for a panel of genes identified as cancer genes. Sixteen (16) candidate genes from the DSBR and cell cycle control pathways were selected for tag SNP-based genotyping (Table 2). A total of 294 SNPs were evaluated for 131 cases and 130 controls that included the subset evaluated in the mutagen sensitivity assay. Forty-four (44) SNPs identified from the 16 candidate genes identified in Table 8 were found to be associated with risk for promoter methylation (p<0.15) with adjustment for covariates. Because of the relatively high correlation between SNPs in these genes, we tested which SNP, or set of SNPs, was most significantly associated with risk for promoter methylation by using a step-wise logistic regression model. The underlined SNPs with p<0.15 from each gene (Table 8) were selected to represent the allelic status for those genes. These 16 SNPs were then included with the covariates in one model and step-wise selection was used to identify the SNPs with the lowest P-value. The minor alleles of ten SNPs from different genes were identified with 4 SNPs associated with increased risk for promoter methylation (ORs, 1.6-4.0) and 6 SNPs with reduced risk for promoter methylation (ORs, 0.4-0.7) (Table 3). Monte Carlo estimates of exact p-values were calculated by permuting the case-control status for all subjects 10,000 times. The exact p-value for five SNPs an allele C in marker rs5762763 of gene CHEK2; an allele T in marker rs7117042 of gene MRE11A; an allele T in marker rs6998169 of gene NBN; an allele A in marker rs7830743 of gene DNA-PKc; an allele C in marker rs2295146 of gene XRCC3 was <0.05 (Table 3). This result indicates that if a similar study were repeated under a null distribution (i.e., no SNPs associated with risk for change in promoter methylation), an association similar to that observed with any of these five SNPs would occur by chance <5% of the time. The underlined SNPs in Table 8 were selected from each gene (p<0.15) to represent the allelic status for those genes. Of the 294 SNPs, 42 SNPs were excluded for data analysis because they were nonpolymorphic or had a minor allele frequency of <0.05 or had a low yield (<80%) or showed a highly significant distortion from Harder Weinberg equilibrium (P<0.001).

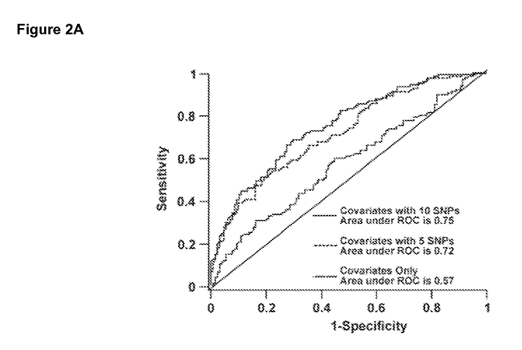

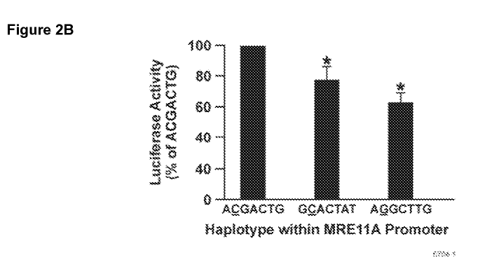

Referring now to FIG. 2, a graph illustrates SNPs in repair genes are associated with gene promoter methylation in sputum and promoter activity of the MRE11A gene. FIG. 2, panel (A) shows ROC curve comparing sensitivity and specificity of SNPs within DNA repair genes for classifying cases and controls. FIG. 2, panel (B) illustrates a difference in MRE11A promoter activity by haplotype whereby the haplotype containing the polymorphic risk marker has the lowest promoter activity. Values are mean±SD from transfection of two constructs containing each haplotype four times and p<0.05 compared to ACGACTG (SEQ ID NO:1).

ROC curves were generated to evaluate the classification accuracy of this panel of SNPs to distinguish cases from controls. The area under the curve increased from 57% (covariates only) to 72% (covariates with the 5 most significant SNPs) and to 75% (covariates with all 10 SNPs, FIG. 2A). The difference between the area under the curve with only covariates and the two models that included both covariates and multiple SNPs is highly significant (p<0.001). Restricting this analysis to include only cases and controls in which DSBRC was determined resulted in an area of 82% that increased to 93% when repair capacity was included in the model. In order to test the hypothesis that the identified SNPs in different genes would work additively to influence risk for promoter methylation, the joint effect of each SNP, inclusive of both putative susceptibility alleles, was evaluated. When the 5 SNPs with the strongest association with risk for promoter methylation were included, persons with 5, 6, or ≧7 alleles out of a total of 10 possible alleles were found to have a 2.5-, 2.8-, and 14.4-fold increased risk, respectively for ≧3 methylated genes from the group comprising p16, MGMT, RASSF1A, PAX5-α, PAX5-β, GATA4, GATA5, and DAPK in sputum compared to those with ≦4 alleles (Table 4).

Reduced activity of the MRE11A promoter. The genes included in the prediction model have biological plausibility, i.e. showing prior association to cancer with respect to sequence variation or activity level. Two of the five genes (MRE11A, NBN, XR CC3, CHEK2, DNA-PKC) whose sequence variation is associated with methylation, NBN and XRCC3, have shown association with lung cancer (Lung Cancer 2005; 49:317-23 and Carcinogenesis 2006; 27: 997-1007). SNPs within the DNA-PKc and CHEK2 genes have been associated with breast and other cancers, while no studies have been conducted with MRE11A (Cancer Res. 2004; 64: 5560-3 and Hum. Mol. Genet. 2007; 16: 1051-7).

Assessment of the functional potential of the SNPs identified from our study for these genes revealed that the minor allele at rs7830743 of DNA-PKC is the polymorphic risk marker and is a nonsynonymous SNP (a SNP that changes the amino acid within a gene sequence) changing amino acid residue 3434 from Ile to Thr in exon 73. This amino acid substitution is predicted to change the secondary structure and may influence the serine/threonine protein kinase activity of this protein Structure 2005; 13: 243-55. We have shown that reduced DNA-PKc activity is associated with risk for lung cancer and sensitivity to cell killing by bleomycin (Carcinogenesis 2001; 22: 723-7), thus supporting an important role for this gene in lung cancer and aberrant gene promoter methylation. The SNPs from the other four genes (CHEK2, XRCC3, DNA-PKc, and NBN) are neither nonsynonymous or in high linkage disequilibrium with any nonsynonymous SNP with known function. However, MRE11A/rs7117042 and NBN/rs6998169 are predicted to locate in the middle of the sequence, forming DNA triplexes that could inhibit DNA transcription (Nucleic Acids Res. 2006; 34: W621-5).

To begin addressing function of these SNPs, we tested whether MRE11A/rs7117042 is associated with a reduction in promoter activity. Referring now to FIG. 2, two subjects homozygous for the haplotype containing MRE11A/rs7117042 and 4 subjects, each homozygous for one of the other four common haplotypes were selected for assessment of MRE11A promoter activity. Sequencing of the 2500 bp promoter of MRE11A revealed three haplotypes ACGACTG (SEQ ID NO:1), GCACTAT (SEQ ID NO 2), and AGGCTTG (SEQ ID NO 3). The three haplotypes were constructed from sequence changes found with the 2500 bp promoter that was sequenced. Of the six subjects whose promoters were sequenced, two of the subjects each have one of the three haplotypes. The most distinct sequence difference was the G to C change at −590 bps. We genotyped 100 subjects selected randomly from our study population for this SNP and found that the G allele was in perfect linkage disequilibrium (R²=1) with the T allele of the polymorphic risk marker rs7117042, identified to be most strongly associated with high methylation index. The promoter region containing each of the three haplotypes was amplified by PCR and cloned into a luciferase reporter assay to measure the effect of each haplotype on activity of the MRE11a promoter. The highest promoter activity was seen in constructs containing the ACGACTG (SEQ ID NO:1) haplotype. With this haplotype as the reference, a 23% and 38% reduction in promoter activity was seen for the GCACTAT (SEQ ID NO:2) and AGGCTTG (SEQ ID NO:3) haplotypes, respectively (FIG. 2B). These results show that the polymorphic risk marker is associated with a marked reduction in transcription of the MRE11A gene.

MRE11A has a role in recognition of double-strand break damage. It complexes with Rad50 and Nbs1 to directly sense the double-strand breaks, binds to the DNA, modifies the ends via 3′ to 5′ exonuclease activity, recruits ATM to the damaged DNA template, and dissociates the ATM dimer (Science 2005; 308: 551-4). Therefore, a reduction in level of the MRE11A protein could have a major impact on DSBRC.

These results indicate a strong link and correlation between reduced DSBRC and risk for methylation in sputum and overall health of a subject.

Another aspect of the present invention provides a method for determining DNA damage that has long been recognized as an initiating event for mutagenesis, or an initiator for initiating aberrant promoter hypermethylation and/or health of a subject.

### Methods

Study population and sample collection. A Smokers Cohort (n=1860) was established in 2001 to conduct longitudinal studies on molecular markers of respiratory carcinogenesis in biological fluids such as sputum from people at risk for lung cancer. At enrollment, individual information about medical, family, and smoking, exposure history, and quality of life was collected through a computer-based system. Induced sputum and blood were collected and pulmonary function testing was performed. Blood was processed within 2 h after blood draw to isolate lymphocytes and plasma. Cryopreservation of lymphocytes began in 2005.

Cytologically adequate sputum samples from 824 cohort subjects were evaluated for gene promoter methylation for a panel of genes comprising (p16, MGMT, DAPK, RASSF1A, PAX5-α, PAX5-β, GATA4 and GATA5). High methylation index was defined as the methylation of three or more gene-specific promoters in sputum. We selected persons from our cohort that exhibited a high (cases) or low (controls [0 of 8 genes]) methylation index. To increase the stringency for case selection, GATA4, which was most commonly methylated in sputum, was excluded as one of the three methylated genes needed for case classification and 131 of 824 cohort subjects met this criteria. Cases were frequency matched by gender to controls. Cases (n=131) and controls (n=130) were selected for the genetic association study. Among the 131 cases, 77 had adequate number of cryopreserved lymphocytes for the mutagen sensitivity assay. Seventy-eight controls were selected from the 130 controls, with frequency matching by gender maintained, for the mutagen sensitivity assay.

Sputum cytology and nested methylation-specific PCR. Sputum samples were stored in Saccomanno's fixative. Three slides were made for each sputum sample to check for adequacy defined as the presence of deep lung macrophages or Curschmann's spiral Diagnostic Pulmonary Cytology 2^{nd} ed. Chicago: Amer. Society of Clinical Pathologists; 1986. The methylation specific PCR assay was only performed on cytologically adequate sputum samples. Eight genes (p16, MGMT, DAPK, RASSF1A, PAX5-α, PAX5-β, GATA4 and GATA5) were selected for analysis of methylation in sputum based on our previous studies establishing their association with risk for lung cancer. Nested MSP was used to detect methylated alleles in DNA recovered from the sputum samples as described (Cancer Res. 2006; 66: 3338-44).

Evaluation of double-strand break repair capacity (DSBRC) in peripheral lymphocytes. PHA-stimulated lymphocytes were treated with bleomycin to evaluate the generation of chromosome aberrations as an index of DSBRC (Int. J. Cancer 1989; 43:403-9). Briefly, cryopreserved lymphocytes were thawed and cultured in RPMI1640 medium supplemented with FBS (20%) and PHA (1.5%) at a cell density of <0.5×10⁶/ml. Sixty-seven hours after PHA stimulation, the culture was split into two T25 flasks and treated with bleomycin or vehicle for 5 h. The final concentration for bleomycin in culture medium was 3 U/l, a concentration defined through dose-response studies using isolated lymphocytes from cohort subjects and two lymphoblastoid cells lines: GM02782 (mutant ATM) and GM00131 (wild-type ATM) (data not shown). The dose selected was within the linear dose-response range and caused obvious genotoxicity, but minimal cytotoxicity. One hour before harvest, colcemid was added to the cultures at a final concentration of 0.06 mg/ml. Slides were prepared according to conventional procedures and 100 well-spread metaphases were examined for chromatid breaks. Samples were assayed as a batch, and slides were scored by a person blinded to case-control status. The criteria of Hsu et al. were used to record the aberrations: a chromatid break was scored as one break and each isochromatid break set was scored as two breaks. Chromosome/chromatid gaps, chromosome-type aberrations (dicentrics, ring, and acentric fragments) or chromatid exchanges were recorded, but not added to the frequencies of chromatid breaks. On rare occasions, a metaphase with >12 breaks was observed on a slide with bleomycin treatment. When this occurred, the number breaks was recorded as 12. The DSBRC was expressed as the mean number of chromatid breaks per cell.

The means of spontaneous chromatid breaks per cell derived from 100 metaphases of untreated cells were 0.013 in cases and 0.021 in controls, which were similar to the spontaneous frequency reported in the literature and < 1/15 the mean number of breaks seen in bleomycin-treated cells (0.32). Therefore, for statistical comparisons, the spontaneous breaks were not subtracted from the breaks observed following treatment with bleomycin.

SNP selection and genotyping by illumina platform for 16 genes in the double-strand break repair pathway and related cell cycle control genes. A total of 294 SNPs were selected for 16 candidate genes from DSBR and cell cycle control pathways Table 9 (Am. J. Hum. Genet. 2004; 74: 106-20 and Bioinformatics 2005; 21: 263-5. Tag SNPs (n=245) were derived from Latino and White data from University of Southern California plus phase 1 HapMap for whites for 15 genes. Tag SNPS were selected using r² ≧0.8 with nonsynonymous SNPs retained as the tag SNPs (Am. J. Hum. Genet. 2004; 74: 106-20). One additional SNP for bins with at least six or more SNPs was selected as a redundant SNP in case of genotyping failure. For the remaining gene, NBN, 49 SNPs were selected using dbSNPs based on a SNP density of 1-3 SNPs/kb depending on the haplotype block structure, validation status, Illumina design score, and functional potential of the SNPs. The number of SNPs selected for each of these 16 genes is shown in Table 2 and Table 9. These SNPs were genotyped by the IIlumina Golden Gate Assay for 261 DNA samples isolated from lymphocytes of cases and controls.

Selection of subjects and construction of MRE11A promoter constructs. Five common haplotypes (6-34%) were constructed based on the 14 tag SNPs assayed for MRE11A in the population (Bioinformatics 2005; 21: 263-5). A Bayesian statistical method implemented in the program PHASE (Version 2.1) was used to reconstruct the haplotypes from the SNPs in the MRE11A gene for the 261 subjects. Two subjects homozygous for the haplotype that contained the RS7117042 SNP associated with high methylation index were selected. The other four people selected were each homozygous for one of the other four haplotypes. The MRE11 promoter fragment (−2541 to −5 with +1 being the translational start site) was amplified from lymphocyte DNA from these six subjects. The promoter fragment was directionally subcloned into the pGL2-basic Luciferase Reporter Vector (Promega, Madison, Wis.) upstream of the luciferase coding sequence. Five clones from each person were commercially sequenced to identify variants within the promoter region (Sequetech, Mountain View, Calif.).

Transient transfection and reporter gene assays. The Calu 6 lung tumor-derived cell line was used for transient transfections. Cells (1.5×10⁵) were plated into 6-well dishes and transfected the following day. Plasmid DNA (1 μg) and the pSV-β Galactosidase control vector (0.5 μg, Promega) were co-transfected into cells with FuGENE 6 transfection reagent (ROCHE Diagnostics, Indianapolis, Ind.) at a FuGENE:DNA ratio of 3:1. A promoter-less pGL2-basic vector and the pGL2-control vector that contains the SV40 promoter were used as negative and positive controls, respectively. Forty-eight hours after transfection, cells were harvested and lysed. Immediately after lysing, cell extracts were assayed in a luminometer for luciferase activity using the Lumionskan Ascent luminometer (Thermo Electron, Milford, Mass.) for luciferase activity using the Luciferase Assay System (Promega). β-galactosidase activity in cell lysates was measured using the Galacto-Star Reporter Gene Assay System (Tropix, Bedford, Mass.). Promoter activity was calculated as the ratio of activities of luciferase and β-galactosidase. Transfections were done in duplicate in four independent experiments.

Statistical analysis. The two-sample t-test, Wilcoxon rank sum test, and x² test were employed to compare the mean or distribution of several demographic variables and DSBRC results between cases and controls as appropriate. Because the DSBRC data and the number of spontaneous breaks were not normally distributed, analysis was also performed on log-transformed data. The results based on log-transformed data were similar to those based on untransformed data so only results based on untransformed data are shown. Analysis of covariance and logistic regression were used to assess the association between selected variables such as SNPs and case-control status, and the outcome variable, DSBRC with adjustment of covariates selected a priori (age at sputum collection, sex, race, current smoking status, and pack years). DSBRC was dichotomized for logistic regression models using the upper quartile of DSBRC in control participants. The selection of the upper quartile of DSBRC in controls as the cut-off value was based on the distribution of DSBRC in cases and controls. Analysis of covariance and logistic regression models, stratified by status were also examined for different associations between SNPs and DSBRC by case-control status. A ROC curve was also drawn to compare the sensitivity and specificity of DSBRC induced by bleomycin for classifying cases (Radiology 1982; 143: 29-36). Multivariate unconditional logistic regression assessed the association between SNPs and the outcome of case-control status, with the same covariates outlined above. Model results are presented as ORs with 95% CIs for having ≧3 methylated genes. Logistic regression modeling was extended to generalized logit models to more precisely examine the high methylation index. ORs and 95% CIs for the risk of having 3, 4 or ≧5 methylated genes with 0 methylated genes as the reference group was obtained with adjustment for the same covariates.

The call rate for each SNP was assessed prior to data analysis. For the 294 SNPs assayed, 42 were deemed unsuitable because they were monomorphic, had MAF<0.05, low yield (<80%), or showed a highly significant distortion from Hardy-Weinberg equilibrium (p<0.0001). These SNPs were removed from analysis. The remaining 252 were analyzed (Table 8). Four models were tested: co-dominant, dominant, additive, and recessive. Because of power limitations, only results for the additive model are presented for each SNP, and common homozygote, heterozygote, and rare homozygote were coded as 0, 1, and 2, respectively. A logistic regression model was used to calculate the ORs and 95% CIs for each individual SNP with adjustment for age, sex, ethnicity, and smoking selected a priori. A ROC curve was drawn to evaluate the classification accuracy of this panel of variables for promoter methylation. An analysis excluding the 23% of study subjects that were not of non-Hispanic white origin had no effect on the identified associations. Therefore, all 261 subjects were included in the data analysis.

Monte Carlo estimates of exact p-values were calculated by permuting the case-control status for all subjects 10,000 times to adjust for multi-comparisons. False positive report probability (FPRP) was also calculated to address the robustness of our findings for individual SNPs (J. Natl. Cancer Inst. 2004; 96: 434-42). In assigning a prior probability for these genes, we considered the strong association between DSBRC and risk for promoter methylation and the stringent r² value (0.8) for selecting tag SNPs. On the basis of the evidence for associations between SNPs in CHEK2, XRCC3, DNA-PKc, NBN, LIG4, and XRCC2 and several cancers, we assigned a relatively high prior probability range (0.1-0.25) for SNPs of these six genes. In contrast, for MRE11A, Ku80, RAD50, and CHEK1, a relatively low prior probability range (0.01-0.1) was assigned because there are no studies that have addressed the association of variants within these genes to cancer. All data analyses were performed with SAS/STAT and SAS/GENETICS 9.1.3.

The expanded regions (100 kb upstream and downstream and coding region) of the top 10 genes downloaded from HapMap project were checked for the SNPs in high LD (r̂2>=0.8) with the risk/protective SNPs reported in Cancer Research 2006; 66: 3338-44. For XRCC2, rs3218400 was not genotyped in HapMap, however, it is in prefect LD (r̂2=1) with rs3218438 in NIEHS EGP project. rs3218438 was genotyped in HapMap. Therefore, both NIEHS and HapMap projects were checked for SNPs in high LD with rs3218400. For the rest of genes in the list, only HapMap database was searched either because small regions were resequenced in NIEHS for some of these genes or unknown ethnicity of population studied. R̂2 reflects the minimal percent agreement between SNPs for linkage disequilibrium.

The present invention has been described in terms of preferred embodiments, however, it will be appreciated that various modifications and improvements may be made to the described embodiments without departing from the scope of the invention. The entire disclosures of all references, applications, patents, and publications cited above and/or in the attachments, and of the corresponding application(s), are hereby incorporated by reference.

TABLE 1
Characteristics of study participants: mutagen sensitivity assay and genetic association study.

| | Cohort members | | | | | | |
|---|---|---|---|---|---|---|---|
| | with methylation | Mutagen sensitivity assay | | | Genetic association study | | |
| Variables | results | Cases | Controls | P value | Cases | Controls | P value |
| Total | 824 | 77 | 78 | | 131 | 130 | |
| Age at enrollment, mean ± SD | 56.7 ± 9.7 | 59.6 ± 9.2 | 55.1 ± 9.4 | 0.003* | 57.2 ± 9.9 | 55.0 ± 9.7 | 0.067* |
| <51 (%) | 33 | 22 | 38 | 0.005† | 31 | 38 | 0.104† |
| 51-63 | 38 | 34 | 41 | | 37 | 41 | |
| ≧63 | 29 | 44 | 21 | | 32 | 21 | |
| Gender (%) | | | | | | | |
| Female | 79 | 62 | 62 | 0.918† | 72 | 72 | 0.921† |
| Male | 21 | 38 | 38 | | 28 | 28 | |
| Race (%) | | | | | | | |
| Non-Hispanic White | 76 | 74 | 73 | 0.510† | 76 | 77 | 0.733† |
| Hispanic | 17 | 17 | 22 | | 18 | 18 | |
| Others | 6 | 9 | 5 | | 7 | 5 | |
| Smoking history | | | | | | | |
| Current (%) | 55 | 47 | 63 | 0.045† | 49 | 57 | 0.172† |
| Pack years, mean ± SD | 40.5 ± 21.5 | 42.8 ± 25.0 | 39.5 ± 22.6 | 0.393* | 42.2 ± 24.3 | 40.4 ± 21.4 | 0.524* |
| Duration, mean ± SD | 33.7 ± 9.7 | 33.4 ± 9.7 | 32.6 ± 8.9 | 0.569* | 33.3 ± 9.9 | 32.9 ± 9.4 | 0.804* |
| Chronic airway obstruction (%)‡ | 26 | 36 | 26 | 0.181† | 36 | 32 | 0.458† |
| Spontaneous chromatid breaks/ | — | 0.013 ± 0.015 | 0.021 ± 0.025 | 0.085§ | | | |
| cell, mean ± SD | | | | | | | |

*Two-sided two-sample t test between cases (methylated group) and controls (unmethylated group)
†χ² test for differences between cases and controls.
‡Chronic airway obstruction is defined as post-bronchodilator FEV1/FVC % <70%.
§Two-sided Wilcoxon rank sum test between cases and controls.

TABLE 2
Number of SNPs in the 16 genes evaluated for association with
gene methylation.

| Gene | No. of SNPs* |
|---|---|
| ATM | 8 |
| ATR | 10 |
| Artemis | 19 |
| CHEK1 | 14 |
| CHEK2 | 12 |
| Ku70 | 6 |
| Ku80 | 25 |
| LIG4 | 11 |
| MRE11 | 14 |
| NBN† | 42 |
| DNA-PKc | 12 |
| RAD50 | 10 |
| TP53 | 8 |
| XRCC2 | 18 |
| XRCC3 | 16 |
| XRCC4 | 27 |
| total | 252 |

*Tag SNPs were selected by pairwise r² method by using Phase I HapMap data for whites and Latino and White data from USC.
†SNPs were selected for NBN using dbSNPs based on the haplotype block structures and the validation status, Illumina design score, and functional potential of SNPs.

TABLE 3
Summary of associations between genes and promoter methylation
using step-wise logistic regression.

| Gene/SNPs* | OR\|\| | 95% CI | P-value | Permuted P-value† |
|---|---|---|---|---|
| MRE11A/rs7117042 | 3.97 | 1.77-8.89 | 0.0008 | 0.0008 |
| CHEK2/rs5762763 | 1.89 | 1.20-2.97 | 0.0064 | 0.0058 |
| XRCC3/rs2295146 | 0.54 | 0.35-0.83 | 0.0051 | 0.0073 |
| DNA-PKc/rs7830743 | 0.38 | 0.18-0.80 | 0.0117 | 0.0142 |
| NBN/rs6998169 | 0.47 | 0.23-0.93 | 0.0452 | 0.0308 |
| LIG4/rs1151402 | 0.68 | 0.44-1.06 | 0.0859 | 0.1078 |
| XRCC2/rs3218400 | 0.55 | 0.28-1.06 | 0.0751 | 0.0823 |
| Ku80/rs828911 | 1.55 | 1.02-2.37 | 0.0416 | 0.059 |
| RAD50/rs2244012 | 1.64 | 0.94-2.76 | 0.0864 | 0.1132 |
| CHEK1/rs537046 | 0.64 | 0.37-1.12 | 0.1176 | 0.1091 |

*Age, sex, ethnicity, smoking status and pack years were selected a priori and forced in the model. Step-wise selection was only used to select genetic susceptibility factors. The p-values for both entry and inclusion of a variable in each round of variable selection were set at 0.1.
†Case and control status was permuted 10,000 times to adjust for multi-comparison.
‡ Statistical power is the power to detect an odds ratio of 2.0 for individual tag SNPs under an additive model.
||ORs were calculated using an additive model where common homozygote, heterozygote, and rare homozygote are coded as 0, 1 and 2, respectively.

TABLE 4
Association between number of risk alleles and promoter
methylation in the Lovelace Smokers Cohort.

| | Cases (%) | Controls (%) | |
|---|---|---|---|
| No. of high-risk alleles | N = 128 | N = 130 | ORs (95% CI)* |
| Top 5 SNPs† | | | |
| ≦4 | 9 (7.0) | 29 (22.3) | 1.00 (reference) |
| 5 | 36 (28.1) | 45 (34.6) | 2.54 (1.06-6.53) |
| 6 | 37 (28.9) | 44 (33.9) | 2.84 (1.18-7.33) |
| ≧7 | 46 (35.9) | 12 (9.2) | 14.39 (5.37-42.45) |
| All 10 SNPs‡ | | | |
| ≦10 | 13 (10.3) | 50 (39.1) | 1.00 (reference) |
| 11 | 26 (20.6) | 26 (20.3) | 4.09 (1.78-9.79) |
| 12 | 31 (24.6) | 34 (26.6) | 3.68 (1.69-8.40) |
| ≧13 | 56 (44.4) | 18 (14.1) | 13.73 (6.08-33.21) |

*Unconditional logistic regression with adjustment for age, sex, ethnicity, smoking status, and pack years.
†Top 5 SNPs include rs7117042, rs5762763, rs2295146, rs7830743 and rs6998169.
‡All 10 SNPs include rs7117042, rs5762763, rs2295146, rs7830743, rs6998169, rs1151402, rs3218400, rs828911, rs2244012 and rs537046.

TABLE 5
Prevalence of gene promoter methylation in sputum
from 824 cohort members.

| | % Positive |
|---|---|
| Gene | |
| p16 | 17.0 |
| MGMT | 22.9 |
| RASSF1A | 1.2 |
| DAPK | 16.3 |
| GATA 4 | 31.2 |
| GATA 5 | 19.9 |
| PAX5-α | 18.7 |
| PAX5-β | 10.8 |
| Number of Genes Methylated | |
| 0 | 32.5 |
| 1 | 28.6 |
| 2 | 19.8 |
| 3 | 10.3 |
| 4 | 5.7 |
| 5 | 2.2 |
| 6 | 0.8 |

TABLE 6
Chromatid break per cell induced by bleomycin between
cases and controls stratified by covariates.

| | Case subjects | | Control subjects | | |
|---|---|---|---|---|---|
| Variables | n | Mean ± SD | n | Mean ± SD | P-value* |
| Total | 77 | 0.473 ± 0.110 | 78 | 0.318 ± 0.098 | <0.0001 |
| Age at sputum | | | | | |
| collection, yr | | | | | |
| <51 | 17 | 0.434 ± 0.061 | 30 | 0.293 ± 0.098 | <0.0001 |
| 51-63 | 26 | 0.465 ± 0.103 | 32 | 0.335 ± 0.095 | <0.0001 |
| ≧63 | 34 | 0.499 ± 0.129 | 16 | 0.329 ± 0.099 | <0.0001 |
| P-value† | | 0.0351 | | 0.0270 | |
| Gender | | | | | |
| Female | 48 | 0.482 ± 0.125 | 48 | 0.318 ± 0.088 | <0.0001 |
| Male | 29 | 0.460 ± 0.081 | 30 | 0.316 ± 0.114 | <0.0001 |
| P-value† | | 0.3690 | | 0.8484 | |
| Race | | | | | |
| Non-Hispanic | 57 | 0.480 ± 0.119 | 57 | 0.311 ± 0.097 | <0.0001 |
| White | | | | | |
| Hispanic | 13 | 0.447 ± 0.040 | 17 | 0.349 ± 0.104 | 0.0017 |
| Others | 7 | 0.473 ± 0.125 | 4 | 0.274 ± 0.062 | 0.0167 |
| P-value† | | 0.6700 | | 0.4187 | |
| Current smoker | | | | | |
| Yes | 36 | 0.474 ± 0.115 | 49 | 0.316 ± 0.093 | <0.0001 |
| No | 41 | 0.473 ± 0.107 | 29 | 0.320 ± 0.107 | <0.0001 |
| P-value† | | 0.3526 | | 0.0694 | |
| Pack years | | | | | |
| <33.2 | 38 | 0.468 ± 0.126 | 37 | 0.315 ± 0.081 | <0.0001 |
| ≧33.2 | 39 | 0.478 ± 0.094 | 41 | 0.320 ± 0.112 | <0.0001 |
| P-value† | | 0.9862 | | 0.7088 | |
| Smoking | | | | | |
| duration, yr | | | | | |
| <34 | 38 | 0.465 ± 0.106 | 40 | 0.306 ± 0.089 | <0.0001 |
| ≧34 | 39 | 0.482 ± 0.117 | 38 | 0.330 ± 0.106 | <0.0001 |
| P-value† | | 0.8654 | | 0.3242 | |
| Chronic airway | | | | | |
| obstruction | | | | | |
| Yes | 28 | 0.489 ± 0.099 | 20 | 0.323 ± 0.089 | <0.0001 |
| No | 49 | 0.464 ± 0.116 | 56 | 0.319 ± 0.102 | <0.0001 |
| P-value† | | 0.4465 | | 0.7346 | |

*indicates two-sided two sample t test between cases and controls.
†Multivariate analysis of covariance with adjustment for age at sputum collection, sex, race, current smoking status, pack years, seeding number of lymphocytes, cryopreservation time, and log-transformed spontaneous chromatid breaks/cell.

TABLE 7
SNPS.

| major | minor | | | | | | | | |
|---|---|---|---|---|---|---|---|---|---|
| allele | allele | SNPs | gene | D′ | r\hat{ }2 | chromosome | coordinate | NCBI build | dbSNP build |
| A | G | rs537046 | Chek1 | | | chr11 | 125015048 | ncbi_b36 | dbSNP b126 |
| G | C | rs535132 | Chek1 | 1.00 | 0.96 | chr11 | 125057793 | ncbi_b36 | dbSNP b126 |
| A | G | rs550323 | Chek1 | 1.00 | 0.96 | chr11 | 125069521 | ncbi_b36 | dbSNP b126 |
| T | C | rs551711 | Chek1 | 1.00 | 0.95 | chr11 | 125072338 | ncbi_b36 | dbSNP b126 |
| C | A | rs526941 | Chek1 | 1.00 | 0.84 | chr11 | 125072749 | ncbi_b36 | dbSNP b126 |
| C | G | rs491071 | Chek1 | 1.00 | 0.96 | chr11 | 125075658 | ncbi_b36 | dbSNP b126 |
| T | C | rs509509 | Chek1 | 1.00 | 0.96 | chr11 | 125075695 | ncbi_b36 | dbSNP b126 |
| G | A | rs536640 | Chek1 | 1.00 | 0.95 | chr11 | 125093925 | ncbi_b36 | dbSNP b126 |
| A | G | rs9613658 | Chek2 | −0.94 | 0.82 | chr22 | 27366465 | ncbi_b36 | dbSNP b126 |
| A | C | rs17415919 | Chek2 | −0.97 | 0.88 | chr22 | 27424828 | ncbi_b36 | dbSNP b126 |
| C | T | rs5762758 | Chek2 | −0.99 | 0.96 | chr22 | 27439036 | ncbi_b36 | dbSNP b126 |
| G | C | rs5762763 | Chek2 | | | chr22 | 27462389 | ncbi_b36 | dbSNP b126 |
| A | G | rs5762764 | Chek2 | −0.95 | 0.90 | chr22 | 27462990 | ncbi_b36 | dbSNP b126 |
| G | C | rs5762765 | Chek2 | 0.95 | 0.87 | chr22 | 27463033 | ncbi_b36 | dbSNP b126 |
| G | A | rs1931348 | Lig4 | −1.00 | 0.97 | chr13 | 107643557 | ncbi_b36 | dbSNP b126 |
| C | T | rs1151402 | Lig4 | | | chr13 | 107656031 | ncbi_b36 | dbSNP b126 |
| T | C | rs1151403 | Lig4 | 1.00 | 1.00 | chr13 | 107656374 | ncbi_b36 | dbSNP b126 |
| A | G | rs1224096 | Lig4 | 0.92 | 0.85 | chr13 | 107701073 | ncbi_b36 | dbSNP b126 |
| C | A | rs10831224 | Mre11a | 1.00 | 0.81 | chr11 | 93785128 | ncbi_b36 | dbSNP b126 |
| A | G | rs13447717 | Mre11a | 1.00 | 0.89 | chr11 | 93809099 | ncbi_b36 | dbSNP b126 |
| C | T | rs7117042 | Mre11a | | | chr11 | 93810623 | ncbi_b36 | dbSNP b126 |
| G | C | rs12222920 | Mre11a | 1.00 | 0.81 | chr11 | 93819763 | ncbi_b36 | dbSNP b126 |
| C | G | rs11020789 | Mre11a | 1.00 | 0.81 | chr11 | 93832834 | ncbi_b36 | dbSNP b126 |
| C | A | rs10831230 | Mre11a | 1.00 | 0.89 | chr11 | 93833296 | ncbi_b36 | dbSNP b126 |
| G | C | rs10831232 | Mre11a | 1.00 | 0.81 | chr11 | 93837973 | ncbi_b36 | dbSNP b126 |
| A | C | rs12224897 | Mre11a | 1.00 | 0.81 | chr11 | 93838730 | ncbi_b36 | dbSNP b126 |
| C | G | rs11825497 | Mre11a | 1.00 | 1.00 | chr11 | 93866205 | ncbi_b36 | dbSNP b129 |
| G | A | rs11020806 | Mre11a | 1.00 | 0.81 | chr11 | 93883620 | ncbi_b36 | dbSNP b126 |
| T | A | rs6998169 | Nbn | | | chr8 | 91019437 | ncbi_b36 | dbSNP b126 |
| T | C | rs10958274 | DNA-PKc | −1.00 | 0.84 | chr8 | 48748528 | ncbi_b36 | dbSNP b126 |
| G | A | rs1487438 | DNA-PKc | −1.00 | 0.84 | chr8 | 48757898 | ncbi_b36 | dbSNP b126 |
| T | A | rs10092880 | DNA-PKc | 1.00 | 1.00 | chr8 | 48791456 | ncbi_b36 | dbSNP b126 |
| A | G | rs7841661 | DNA-PKc | 1.00 | 1.00 | chr8 | 48805431 | ncbi_b36 | dbSNP b126 |
| A | T | rs3614 | DNA-PKc | 1.00 | 1.00 | chr8 | 48811243 | ncbi_b36 | dbSNP b126 |
| A | C | rs9918758 | DNA-PKc | 1.00 | 1.00 | chr8 | 48826267 | ncbi_b36 | dbSNP b126 |
| G | A | rs7830633 | DNA-PKc | 1.00 | 1.00 | chr8 | 48840758 | ncbi_b36 | dbSNP b126 |
| C | G | rs7839161 | DNA-PKc | 1.00 | 1.00 | chr8 | 48846908 | ncbi_b36 | dbSNP b126 |
| C | T | rs8178258 | DNA-PKc | 1.00 | 1.00 | chr8 | 48851919 | ncbi_b36 | dbSNP b126 |
| A | G | rs8178255 | DNA-PKc | 1.00 | 1.00 | chr8 | 48852619 | ncbi_b36 | dbSNP b126 |
| C | G | rs8178238 | DNA-PKc | 1.00 | 1.00 | chr8 | 48859977 | ncbi_b36 | dbSNP b126 |
| A | G | rs6995756 | DNA-PKc | 1.00 | 1.00 | chr8 | 48872854 | ncbi_b36 | dbSNP b126 |
| A | G | rs7830743 | DNA-PKc | | | chr8 | 48873508 | ncbi_b36 | dbSNP b126 |
| C | G | rs7828380 | DNA-PKc | 1.00 | 1.00 | chr8 | 48874235 | ncbi_b36 | dbSNP b126 |
| C | T | rs7838910 | DNA-PKc | 1.00 | 1.00 | chr8 | 48876888 | ncbi_b36 | dbSNP b126 |
| T | C | rs7832898 | DNA-PKc | 1.00 | 1.00 | chr8 | 48885336 | ncbi_b36 | dbSNP b126 |
| G | T | rs7818445 | DNA-PKc | 1.00 | 1.00 | chr8 | 48887689 | ncbi_b36 | dbSNP b126 |
| C | A | rs4873728 | DNA-PKc | −1.00 | 0.91 | chr8 | 48901993 | ncbi_b36 | dbSNP b126 |
| C | G | rs7014544 | DNA-PKc | 1.00 | 1.00 | chr8 | 48907538 | ncbi_b36 | dbSNP b126 |
| C | T | rs10097508 | DNA-PKc | 1.00 | 1.00 | chr8 | 48907897 | ncbi_b36 | dbSNP b126 |
| T | C | rs8178169 | DNA-PKc | 1.00 | 1.00 | chr8 | 48927642 | ncbi_b36 | dbSNP b126 |
| T | G | rs4873737 | DNA-PKc | 1.00 | 1.00 | chr8 | 48928838 | ncbi_b36 | dbSNP b126 |
| C | G | rs8178158 | DNA-PKc | 1.00 | 1.00 | chr8 | 48930071 | ncbi_b36 | dbSNP b126 |
| C | T | rs6993483 | DNA-PKc | 1.00 | 1.00 | chr8 | 48938742 | ncbi_b36 | dbSNP b126 |
| G | A | rs8178095 | DNA-PKc | 1.00 | 1.00 | chr8 | 48964599 | ncbi_b36 | dbSNP b126 |
| G | A | rs10097783 | DNA-PKc | −1.00 | 0.83 | chr8 | 48968135 | ncbi_b36 | dbSNP b126 |
| G | A | rs12334811 | DNA-PKc | 1.00 | 1.00 | chr8 | 48995530 | ncbi_b36 | dbSNP b126 |
| T | C | rs10106778 | DNA-PKc | 1.00 | 1.00 | chr8 | 48995839 | ncbi_b36 | dbSNP b126 |
| C | T | rs4873770 | DNA-PKc | 1.00 | 1.00 | chr8 | 49008811 | ncbi_b36 | dbSNP b126 |
| C | T | rs8178016 | DNA-PKc | 1.00 | 1.00 | chr8 | 49014881 | ncbi_b36 | dbSNP b126 |
| A | C | rs1551655 | DNA-PKc | 1.00 | 1.00 | chr8 | 49035814 | ncbi_b36 | dbSNP b126 |
| C | T | rs9657054 | DNA-PKc | 1.00 | 1.00 | chr8 | 49045452 | ncbi_b36 | dbSNP b126 |
| T | C | rs1894311 | DNA-PKc | 1.00 | 1.00 | chr8 | 49047602 | ncbi_b36 | dbSNP b126 |
| G | A | rs4873266 | DNA-PKc | 1.00 | 1.00 | chr8 | 49072277 | ncbi_b36 | dbSNP b126 |
| T | G | rs28641816 | DNA-PKc | 1.00 | 1.00 | chr8 | 49098664 | ncbi_b36 | dbSNP b126 |
| G | C | rs12652920 | Rad50 | 1.00 | 1.00 | chr5 | 131913139 | ncbi_b36 | dbSNP b126 |
| C | T | rs2706338 | Rad50 | 1.00 | 0.98 | chr5 | 131923748 | ncbi_b36 | dbSNP b126 |
| A | G | rs2244012 | Rad50 | | | chr5 | 131929124 | ncbi_b36 | dbSNP b126 |
| T | G | rs2299015 | Rad50 | 1.00 | 1.00 | chr8 | 131929396 | ncbi_b36 | dbSNP b126 |
| G | T | rs2706347 | Rad50 | 1.00 | 1.00 | chr5 | 131933016 | ncbi_b36 | dbSNP b126 |
| G | A | rs2706348 | Rad50 | 1.00 | 1.00 | chr8 | 131933709 | ncbi_b36 | dbSNP b126 |
| G | A | rs17166050 | Rad50 | 1.00 | 1.00 | chr8 | 131943112 | ncbi_b36 | dbSNP b126 |
| T | C | rs2522403 | Rad50 | 1.00 | 1.00 | chr8 | 131943216 | ncbi_b36 | dbSNP b126 |
| A | G | rs2246176 | Rad50 | 1.00 | 1.00 | chr8 | 131945249 | ncbi_b36 | dbSNP b126 |
| T | G | rs2252775 | Rad50 | 1.00 | 1.00 | chr8 | 131946343 | ncbi_b36 | dbSNP b126 |
| T | C | rs10463893 | Rad50 | 1.00 | 1.00 | chr8 | 131955938 | ncbi_b36 | dbSNP b126 |
| G | T | rs2897443 | Rad50 | 1.00 | 1.00 | chr8 | 131957493 | ncbi_b36 | dbSNP b126 |
| G | A | rs17622991 | Rad50 | −1.00 | 0.90 | chr8 | 131960652 | ncbi_b36 | dbSNP b126 |
| A | C | rs2706370 | Rad50 | 1.00 | 0.92 | chr8 | 131960915 | ncbi_b36 | dbSNP b126 |
| C | T | rs2706372 | Rad50 | 1.00 | 1.00 | chr8 | 131963376 | ncbi_b36 | dbSNP b126 |
| T | G | rs12187537 | Rad50 | 1.00 | 1.00 | chr8 | 131967803 | ncbi_b36 | dbSNP b126 |
| G | A | rs2522394 | Rad50 | 1.00 | 1.00 | chr8 | 131972028 | ncbi_b36 | dbSNP b126 |
| A | G | rs10520114 | Rad50 | 1.00 | 1.00 | chr8 | 131976790 | ncbi_b36 | dbSNP b126 |
| T | C | rs2301713 | Rad50 | 1.00 | 1.00 | chr8 | 131979895 | ncbi_b36 | dbSNP b126 |
| T | C | rs6596086 | Rad50 | 1.00 | 1.00 | chr8 | 131980121 | ncbi_b36 | dbSNP b126 |
| T | A | rs2106984 | Rad50 | 1.00 | 1.00 | chr8 | 131980965 | ncbi_b36 | dbSNP b126 |
| C | T | rs7449456 | Rad50 | 1.00 | 1.00 | chr8 | 131981326 | ncbi_b36 | dbSNP b126 |
| C | T | rs3798135 | Rad50 | 1.00 | 1.00 | chr8 | 131993008 | ncbi_b36 | dbSNP b126 |
| G | A | rs3798134 | Rad50 | 1.00 | 1.00 | chr8 | 131993078 | ncbi_b36 | dbSNP b126 |
| G | A | rs6596087 | Rad50 | 1.00 | 1.00 | chr8 | 131996508 | ncbi_b36 | dbSNP b126 |
| T | C | rs6871536 | Rad50 | 1.00 | 1.00 | chr8 | 131997773 | ncbi_b36 | dbSNP b126 |
| C | T | rs12653750 | Rad50 | 1.00 | 1.00 | chr8 | 131999801 | ncbi_b36 | dbSNP b126 |
| C | G | rs2040703 | Rad50 | 1.00 | 1.00 | chr8 | 132000157 | ncbi_b36 | dbSNP b126 |
| A | G | rs2040704 | Rad50 | 1.00 | 1.00 | chr8 | 132001076 | ncbi_b36 | dbSNP b126 |
| T | C | rs2074369 | Rad50 | 1.00 | 1.00 | chr8 | 132001562 | ncbi_b36 | dbSNP b126 |
| T | A | rs7737470 | Rad50 | 1.00 | 1.00 | chr8 | 132001962 | ncbi_b36 | dbSNP b126 |
| C | T | rs2240032 | Rad50 | 1.00 | 1.00 | chr8 | 132005026 | ncbi_b36 | dbSNP b126 |
| A | G | rs2158177 | Rad50 | 1.00 | 0.91 | chr8 | 132011957 | ncbi_b36 | dbSNP b126 |
| A | G | rs3091307 | Rad50 | 1.00 | 1.00 | chr5 | 132017035 | ncbi_b36 | dbSNP b126 |
| A | C | rs1881457 | Rad50 | 1.00 | 0.91 | chr5 | 132020308 | ncbi_b36 | dbSNP b126 |
| G | A | rs3218489 | Xrcc2 | 1.00 | 0.88 | chr7 | 151985036 | ncbi_b36 | dbSNP b126 |
| TGTT | — | rs3218478 | Xrcc2 | 1.00 | 1.00 | chr7 | 151987970 | ncbi_b36 | dbSNP b126 |
| A | G | rs3218438 | Xrcc2 | 1.00 | 1.00 | chr7 | 151994159 | ncbi_b36 | dbSNP b126 |
| C | A | rs3218400 | Xrcc2 | | | chr7 | 152000622 | ncbi_b36 | dbSNP b126 |
| G | A | rs941474 | Xrcc3 | −1.00 | 0.82 | chr14 | 103259614 | ncbi_b36 | dbSNP b126 |
| C | G | rs2295151 | Xrcc3 | 1.00 | 0.86 | chr14 | 103262852 | ncbi_b36 | dbSNP b126 |
| T | C | rs2295148 | Xrcc3 | −1.00 | 0.83 | chr14 | 103265363 | ncbi_b36 | dbSNP b126 |
| C | T | rs2295147 | Xrcc3 | 1.00 | 0.84 | chr14 | 103265417 | ncbi_b36 | dbSNP b126 |
| G | A | rs12433109 | Xrcc3 | −0.99 | 0.96 | chr14 | 103266360 | ncbi_b36 | dbSNP b126 |
| T | C | rs3742365 | Xrcc3 | −1.00 | 0.97 | chr14 | 103268004 | ncbi_b36 | dbSNP b126 |
| C | T | rs2295146 | Xrcc3 | | | chr14 | 103269109 | ncbi_b36 | dbSNP b126 |
| G | A | rs2295145 | Xrcc3 | −0.96 | 0.88 | chr14 | 103272057 | ncbi_b36 | dbSNP b126 |
| G | A | rs8004408 | Xrcc3 | −0.96 | 0.86 | chr14 | 103273691 | ncbi_b36 | dbSNP b126 |
| G | A | rs7156834 | Xrcc3 | −0.95 | 0.85 | chr14 | 103280933 | ncbi_b36 | dbSNP b126 |
| T | C | rs2295141 | Xrcc3 | −0.96 | 0.88 | chr14 | 103282702 | ncbi_b36 | dbSNP b126 |
| C | T | rs1997913 | Xrcc3 | 0.96 | 0.85 | chr14 | 103283713 | ncbi_b36 | dbSNP b126 |
| C | T | rs3818085 | Xrcc3 | 0.96 | 0.88 | chr14 | 103285738 | ncbi_b36 | dbSNP b126 |
| T | A | rs1535098 | Xrcc3 | −0.94 | 0.86 | chr14 | 103286266 | ncbi_b36 | dbSNP b126 |
| C | T | rs1535097 | Xrcc3 | 0.96 | 0.88 | chr14 | 103286402 | ncbi_b36 | dbSNP b126 |
| G | A | rs11847468 | Xrcc3 | −0.96 | 0.88 | chr14 | 103290048 | ncbi_b36 | dbSNP b126 |
| C | A | rs11625740 | Xrcc3 | −0.95 | 0.85 | chr14 | 103291559 | ncbi_b36 | dbSNP b126 |
| A | G | rs6575997 | Xrcc3 | 0.96 | 0.87 | chr14 | 103298299 | ncbi_b36 | dbSNP b126 |
| G | C | rs4906365 | Xrcc3 | −0.95 | 0.85 | chr14 | 103298983 | ncbi_b36 | dbSNP b126 |
| C | T | rs11160759 | Xrcc3 | 0.95 | 0.85 | chr14 | 103301295 | ncbi_b36 | dbSNP b126 |
| G | A | rs11626377 | Xrcc3 | −0.95 | 0.85 | chr14 | 103304110 | ncbi_b36 | dbSNP b126 |
| G | A | rs876002 | Xrcc3 | −0.96 | 0.88 | chr14 | 103309450 | ncbi_b36 | dbSNP b126 |
| T | C | rs11624184 | Xrcc3 | −0.96 | 0.86 | chr14 | 103310751 | ncbi_b36 | dbSNP b126 |
| C | T | rs11628332 | Xrcc3 | 0.96 | 0.88 | chr14 | 103310894 | ncbi_b36 | dbSNP b126 |
| G | T | rs11160760 | Xrcc3 | 0.96 | 0.88 | chr14 | 103318691 | ncbi_b36 | dbSNP b126 |
| C | T | rs4900594 | Xrcc3 | 0.95 | 0.86 | chr14 | 103320759 | ncbi_b36 | dbSNP b126 |
| G | A | rs11160762 | Xrcc3 | −0.96 | 0.88 | chr14 | 103324836 | ncbi_b36 | dbSNP b126 |
| G | A | rs7147171 | Xrcc3 | −0.96 | 0.86 | chr14 | 103334610 | ncbi_b36 | dbSNP b126 |
| A | G | rs11623546 | Xrcc3 | 0.93 | 0.85 | chr14 | 103338851 | ncbi_b36 | dbSNP b126 |
| G | T | rs12879501 | Xrcc3 | 0.91 | 0.81 | chr14 | 103342387 | ncbi_b36 | dbSNP b126 |
| C | T | rs2368560 | Xrcc3 | 0.90 | 0.80 | chr14 | 103343064 | ncbi_b36 | dbSNP b126 |
| A | G | rs12885018 | Xrcc3 | 0.90 | 0.80 | chr14 | 103343565 | ncbi_b36 | dbSNP b126 |
| T | C | rs12891175 | Xrcc3 | −0.96 | 0.87 | chr14 | 103343919 | ncbi_b36 | dbSNP b126 |
| A | G | rs12889993 | Xrcc3 | 0.96 | 0.87 | chr14 | 103344047 | ncbi_b36 | dbSNP b126 |
| C | T | rs12880821 | Xrcc3 | 0.95 | 0.85 | chr14 | 103345802 | ncbi_b36 | dbSNP b126 |
| T | C | rs8005594 | Xrcc3 | −0.95 | 0.85 | chr14 | 103349642 | ncbi_b36 | dbSNP b126 |
| T | C | rs2887282 | Xrcc3 | −0.96 | 0.88 | chr14 | 103350487 | ncbi_b36 | dbSNP b126 |
| T | C | rs11898924 | KU80 | 1.00 | 0.95 | chr2 | 216659966 | ncbi_b36 | dbSNP b126 |
| C | T | rs6736096 | KU80 | 1.00 | 0.98 | chr2 | 216666614 | ncbi_b36 | dbSNP b126 |
| T | C | rs1344600 | KU80 | 1.00 | 0.98 | chr2 | 216669766 | ncbi_b36 | dbSNP b126 |
| C | T | rs6730091 | KU80 | 1.00 | 0.82 | chr2 | 216679046 | ncbi_b36 | dbSNP b126 |
| G | T | rs828907 | KU80 | 1.00 | 1.00 | chr2 | 216680977 | ncbi_b36 | dbSNP b126 |
| A | G | rs828909 | KU80 | 1.00 | 0.80 | chr2 | 216683550 | ncbi_b36 | dbSNP b126 |
| G | A | rs828910 | KU80 | 1.00 | 0.82 | chr2 | 216685273 | ncbi_b36 | dbSNP b126 |
| G | A | rs828911 | KU80 | | | chr2 | 216685568 | ncbi_b36 | dbSNP b126 |
| T | G | rs828703 | KU80 | 1.00 | 0.89 | chr2 | 216701787 | ncbi_b36 | dbSNP b126 |
| T | C | rs207876 | KU80 | 0.99 | 0.86 | chr2 | 216704832 | ncbi_b36 | dbSNP b126 |
| A | G | rs207878 | KU80 | −0.99 | 0.86 | chr2 | 216706906 | ncbi_b36 | dbSNP b126 |

TABLE 8
Individual SNPs associated with risk for promoter methylation
at p values ≦0.15 for the 16 genes evaluated in this study.

| rs_num* | Chr | Gene† | Allele | MAF | P-value | ORs | low | high |
|---|---|---|---|---|---|---|---|---|
| rs7913426 | chr10 | Artemis | G | 0.13 | 0.044 | 0.56 | 0.31 | 0.97 |
| rs7476111 | chr10 | Artemis | T | 0.33 | 0.138 | 1.32 | 0.92 | 1.91 |
| rs584531 | chr11 | MRE11 | C | 0.39 | 0.058 | 0.70 | 0.48 | 1.01 |
| rs2508678 | chr11 | MRE11 | T | 0.32 | 0.131 | 1.34 | 0.92 | 1.97 |
| rs7117042 | chr11 | MRE11 | T | 0.05 | 0.002 | 3.00 | 1.51 | 6.28 |
| rs604845 | chr11 | MRE11 | T | 0.39 | 0.038 | 0.68 | 0.47 | 0.98 |
| rs533984 | chr11 | MRE11 | A | 0.34 | 0.092 | 1.37 | 0.95 | 1.98 |
| rs540199 | chr11 | MRE11 | G | 0.37 | 0.144 | 0.76 | 0.53 | 1.10 |
| rs1801516 | chr11 | ATM | A | 0.09 | 0.062 | 1.74 | 0.98 | 3.16 |
| rs373759 | chr11 | ATM | T | 0.41 | 0.110 | 0.73 | 0.50 | 1.07 |
| rs540723 | chr11 | CHEK1 | A | 0.10 | 0.082 | 1.62 | 0.95 | 2.82 |
| rs537046 | chr11 | CHEK1 | G | 0.16 | 0.075 | 0.64 | 0.39 | 1.04 |
| rs9514825 | chr13 | LIG4 | T | 0.36 | 0.075 | 1.38 | 0.97 | 1.98 |
| rs4635191 | chr13 | LIG4 | G | 0.25 | 0.107 | 1.37 | 0.94 | 2.01 |
| rs1151402 | chr13 | LIG4 | T | 0.45 | 0.045 | 0.67 | 0.45 | 0.99 |
| rs2273175 | chr14 | XRCC3 | C | 0.41 | 0.089 | 0.73 | 0.51 | 1.05 |
| rs2295148 | chr14 | XRCC3 | T | 0.47 | 0.061 | 0.70 | 0.49 | 1.01 |
| rs2295146 | chr14 | XRCC3 | T | 0.47 | 0.015 | 0.63 | 0.43 | 0.91 |
| rs8548 | chr14 | XRCC3 | C | 0.40 | 0.097 | 0.73 | 0.51 | 1.06 |
| rs3825550 | chr14 | XRCC3 | T | 0.03 | 0.096 | 2.25 | 0.89 | 6.19 |
| rs828910 | chr2 | Ku80 | G | 0.46 | 0.108 | 1.35 | 0.94 | 1.96 |
| rs828911 | chr2 | Ku80 | A | 0.41 | 0.093 | 1.37 | 0.95 | 1.98 |
| rs828701 | chr2 | Ku80 | T | 0.45 | 0.066 | 1.40 | 0.98 | 2.02 |
| rs2303400 | chr2 | Ku80 | C | 0.46 | 0.113 | 0.74 | 0.50 | 1.07 |
| rs207908 | chr2 | Ku80 | T | 0.43 | 0.105 | 1.37 | 0.94 | 2.01 |
| rs5752776 | chr22 | CHEK2 | A | 0.34 | 0.099 | 0.73 | 0.49 | 1.06 |
| rs9620817 | chr22 | CHEK2 | T | 0.13 | 0.067 | 0.56 | 0.29 | 1.03 |
| rs5762763 | chr22 | CHEK2 | C | 0.30 | 0.023 | 1.58 | 1.07 | 2.36 |
| rs2236141 | chr22 | CHEK2 | T | 0.12 | 0.032 | 1.76 | 1.06 | 2.99 |
| rs6519265 | chr22 | Ku70 | A | 0.20 | 0.114 | 1.39 | 0.93 | 2.12 |
| rs132793 | chr22 | Ku70 | A | 0.20 | 0.091 | 1.43 | 0.95 | 2.17 |
| rs10804682 | chr3 | ATR | A | 0.22 | 0.094 | 0.67 | 0.41 | 1.07 |
| rs2244012 | chr5 | RAD50 | G | 0.18 | 0.135 | 1.43 | 0.90 | 2.29 |
| rs6596087 | chr5 | RAD50 | A | 0.18 | 0.112 | 1.46 | 0.92 | 2.34 |
| rs6871536 | chr5 | RAD50 | C | 0.18 | 0.138 | 1.42 | 0.90 | 2.27 |
| rs3218400 | chr7 | XRCC2 | T | 0.12 | 0.123 | 0.62 | 0.33 | 1.13 |
| rs7830743 | chr8 | DNA-PKc | G | 0.11 | 0.071 | 0.54 | 0.27 | 1.04 |
| rs4873737 | chr8 | DNA-PKc | G | 0.12 | 0.080 | 0.57 | 0.30 | 1.06 |
| rs4873772 | chr8 | DNA-PKc | A | 0.31 | 0.128 | 1.34 | 0.92 | 1.97 |
| rs10091017 | chr8 | DNA-PKc | A | 0.10 | 0.062 | 0.52 | 0.26 | 1.02 |
| rs14448 | chr8 | NBN | G | 0.06 | 0.064 | 1.91 | 0.98 | 3.87 |
| rs9995 | chr8 | NBN | G | 0.36 | 0.102 | 0.73 | 0.49 | 1.06 |
| SB_rs1063054 | chr8 | NBN | G | 0.36 | 0.130 | 0.74 | 0.50 | 1.09 |
| SB_rs2735383 | chr8 | NBN | G | 0.36 | 0.141 | 0.75 | 0.51 | 1.10 |
| rs6998169 | chr8 | NBN | A | 0.13 | 0.014 | 0.45 | 0.23 | 0.84 |

†XRCC2 and XRCC4 were not listed in this table because of no SNPs showing association with methylation at p value less than 0.15.

TABLE 9
SNPs

| | | | | NCBI | dbSNP |
|---|---|---|---|---|---|
| SNP_Name | Chr | Gene | Coordinate | build | build |
| rs609557 | chr11 | ATM | 107589723 | 36 | 128 |
| rs228608 | chr11 | ATM | 107594110 | 36 | 128 |
| rs4987876 | chr11 | ATM | 107597847 | 36 | 128 |
| rs228590 | chr11 | ATM | 107601351 | 36 | 128 |
| rs228595 | chr11 | ATM | 107610803 | 36 | 128 |
| rs2234997 | chr11 | ATM | 107611653 | 36 | 128 |
| rs4986761 | chr11 | ATM | 107629971 | 36 | 128 |
| rs1800057 | chr11 | ATM | 107648666 | 36 | 128 |
| rs1800058 | chr11 | ATM | 107665560 | 36 | 128 |
| rs1800889 | chr11 | ATM | 107668697 | 36 | 128 |
| rs1801516 | chr11 | ATM | 107680672 | 36 | 128 |
| rs373759 | chr11 | ATM | 107725867 | 36 | 128 |
| rs227094 | chr11 | ATM | 107739010 | 36 | 128 |
| rs11719737 | chr3 | ATR | 143646707 | 36 | 128 |
| rs1802904 | chr3 | ATR | 143651021 | 36 | 128 |
| rs2229032 | chr3 | ATR | 143660834 | 36 | 128 |
| rs4582075 (merged | chr3 | ATR | 143676235 | 36 | 128 |
| from rs7431240) | | | | | |
| rs6805118 | chr3 | ATR | 143699606 | 36 | 128 |
| rs10804682 | chr3 | ATR | 143717224 | 36 | 128 |
| rs7636909 | chr3 | ATR | 143743252 | 36 | 128 |
| rs13091637 | chr3 | ATR | 143749129 | 36 | 128 |
| rs2229033 | chr3 | ATR | 143764043 | 36 | 128 |
| rs7632782 | chr3 | ATR | 143795088 | 36 | 128 |
| rs6440092 | chr3 | ATR | 143796677 | 36 | 128 |
| rs6414350 | chr3 | ATR | 143799350 | 36 | 128 |
| rs7907802 | chr10 | Artemis | 14980700 | 36 | 128 |
| rs11594111 | chr10 | Artemis | 14985412 | 36 | 128 |
| rs7921238 | chr10 | Artemis | 14991711 | 36 | 128 |
| rs7922341 | chr10 | Artemis | 14992798 | 36 | 128 |
| rs12572872 | chr10 | Artemis | 14994030 | 36 | 128 |
| rs10906777 | chr10 | Artemis | 15002870 | 36 | 128 |
| rs10128350 | chr10 | Artemis | 15003678 | 36 | 128 |
| rs2066325 | chr10 | Artemis | 15007411 | 36 | 128 |
| rs2004392 | chr10 | Artemis | 15020333 | 36 | 128 |
| rs7916722 | chr10 | Artemis | 15020687 | 36 | 128 |
| rs7913426 | chr10 | Artemis | 15020768 | 36 | 128 |
| rs10796227 | chr10 | Artemis | 15021542 | 36 | 128 |
| rs7920514 | chr10 | Artemis | 15027446 | 36 | 128 |
| rs7916726 | chr10 | Artemis | 15030375 | 36 | 128 |
| rs7906967 | chr10 | Artemis | 15031436 | 36 | 128 |
| rs6602769 | chr10 | Artemis | 15032934 | 36 | 128 |
| rs4360596 (merged | chr10 | Artemis | 15043083 | 36 | 128 |
| from rs7476111) | | | | | |
| rs7919322 | chr10 | Artemis | 15043284 | 36 | 128 |
| rs10906785 | chr10 | Artemis | 15046525 | 36 | 128 |
| rs2298113 | chr10 | Artemis | 15052367 | 36 | 128 |
| rs2298112 | chr10 | Artemis | 15052541 | 36 | 128 |
| rs12259856 | chr10 | Artemis | 15055296 | 36 | 128 |
| rs3740901 | chr11 | CHEK1 | 124981753 | 36 | 128 |
| rs477961 | chr11 | CHEK1 | 124982588 | 36 | 128 |
| rs2241502 | chr11 | CHEK1 | 124984573 | 36 | 128 |
| rs2241501 | chr11 | CHEK1 | 124984706 | 36 | 128 |
| rs11220159 | chr11 | CHEK1 | 124988381 | 36 | 128 |
| rs540723 | chr11 | CHEK1 | 124994831 | 36 | 128 |
| rs525186 | chr11 | CHEK1 | 124994998 | 36 | 128 |
| rs491741 | chr11 | CHEK1 | 124999089 | 36 | 128 |
| rs2298483 | chr11 | CHEK1 | 125000232 | 36 | 128 |
| rs3731422 | chr11 | CHEK1 | 125012487 | 36 | 128 |
| rs537046 | chr11 | CHEK1 | 125015048 | 36 | 128 |
| rs3731438 | chr11 | CHEK1 | 125016423 | 36 | 128 |
| rs506504 | chr11 | CHEK1 | 125030405 | 36 | 128 |
| rs7940584 | chr11 | CHEK1 | 125032999 | 36 | 128 |
| rs519772 | chr11 | CHEK1 | 125033601 | 36 | 128 |
| rs6005835 | chr22 | CHEK2 | 27405319 | 36 | 128 |
| rs2267130 | chr22 | CHEK2 | 27429754 | 36 | 128 |
| rs6519761 | chr22 | CHEK2 | 27431600 | 36 | 128 |
| rs2073327 | chr22 | CHEK2 | 27435558 | 36 | 128 |
| rs1884817 | chr22 | CHEK2 | 27436945 | 36 | 128 |
| rs5752776 | chr22 | CHEK2 | 27438229 | 36 | 128 |
| rs9620817 | chr22 | CHEK2 | 27438556 | 36 | 128 |
| rs5762763 | chr22 | CHEK2 | 27462389 | 36 | 128 |
| rs5762766 (merged | chr22 | CHEK2 | 27465889 | 36 | 128 |
| from rs10854805) | | | | | |
| rs2236141 | chr22 | CHEK2 | 27467870 | 36 | 128 |
| rs2236142 | chr22 | CHEK2 | 27467944 | 36 | 128 |
| rs5752791 | chr22 | CHEK2 | 27483547 | 36 | 128 |
| rs9306460 | chr22 | CHEK2 | 27486170 | 36 | 128 |
| rs4873672 | chr8 | DNA_PKc | 48857074 | 36 | 128 |
| rs7830743 | chr8 | DNA_PKc | 48873508 | 36 | 128 |
| rs8178215 | chr8 | DNA_PKc | 48892104 | 36 | 128 |
| rs4521758 | chr8 | DNA_PKc | 48904123 | 36 | 128 |
| rs4873737 | chr8 | DNA_PKc | 48928838 | 36 | 128 |
| rs7003908 | chr8 | DNA_PKc | 48933255 | 36 | 128 |
| rs8178148 | chr8 | DNA_PKc | 48933854 | 36 | 128 |
| rs8178129 | chr8 | DNA_PKc | 48936726 | 36 | 128 |
| rs10109984 | chr8 | DNA_PKc | 48966228 | 36 | 128 |
| rs8178071 | chr8 | DNA_PKc | 48977140 | 36 | 128 |
| rs2213178 | chr8 | DNA_PKc | 48979269 | 36 | 128 |
| rs3829985 | chr8 | DNA_PKc | 49002653 | 36 | 128 |
| rs1231201 | chr8 | DNA_PKc | 49008716 | 36 | 128 |
| rs8178017 | chr8 | DNA_PKc | 49014778 | 36 | 128 |
| rs4873772 | chr8 | DNA_PKc | 49021486 | 36 | 128 |
| rs762679 | chr8 | DNA_PKc | 49047989 | 36 | 128 |
| rs10091017 | chr8 | DNA_PKc | 49049023 | 36 | 128 |
| rs2267437 | chr22 | Ku70 | 40346645 | 36 | 128 |
| rs132770 | chr22 | Ku70 | 40347210 | 36 | 128 |
| rs132771 (merged | chr22 | Ku70 | 40355296 | 36 | 128 |
| from rs6519265) | | | | | |
| rs132788 | chr22 | Ku70 | 40389714 | 36 | 128 |
| rs11703638 | chr22 | Ku70 | 40392713 | 36 | 128 |
| rs132793 | chr22 | Ku70 | 40393627 | 36 | 128 |
| rs828920 | chr2 | Ku80 | 216663580 | 36 | 128 |
| rs828922 | chr2 | Ku80 | 216664913 | 36 | 128 |
| rs1425118 | chr2 | Ku80 | 216667144 | 36 | 128 |
| rs10498045 | chr2 | Ku80 | 216669933 | 36 | 128 |
| rs828910 | chr2 | Ku80 | 216685273 | 36 | 128 |
| rs828911 | chr2 | Ku80 | 216685568 | 36 | 128 |
| rs3815855 | chr2 | Ku80 | 216690615 | 36 | 128 |
| rs10166817 | chr2 | Ku80 | 216690936 | 36 | 128 |
| rs828701 | chr2 | Ku80 | 216699216 | 36 | 128 |
| rs828702 | chr2 | Ku80 | 216701723 | 36 | 128 |
| rs1805382 | chr2 | Ku80 | 216703836 | 36 | 128 |
| rs2303400 | chr2 | Ku80 | 216711397 | 36 | 128 |
| rs207905 | chr2 | Ku80 | 216719857 | 36 | 128 |
| rs207908 | chr2 | Ku80 | 216724192 | 36 | 128 |
| rs207910 | chr2 | Ku80 | 216726667 | 36 | 128 |
| rs207916 | chr2 | Ku80 | 216735805 | 36 | 128 |
| rs3821107 | chr2 | Ku80 | 216739573 | 36 | 128 |
| rs207922 | chr2 | Ku80 | 216739754 | 36 | 128 |
| rs207928 | chr2 | Ku80 | 216744686 | 36 | 128 |
| rs207939 | chr2 | Ku80 | 216750743 | 36 | 128 |
| rs3770497 | chr2 | Ku80 | 216755885 | 36 | 128 |
| rs3770494 | chr2 | Ku80 | 216758262 | 36 | 128 |
| rs2241320 | chr2 | Ku80 | 216762758 | 36 | 128 |
| rs1051685 | chr2 | Ku80 | 216778621 | 36 | 128 |
| rs207884 | chr2 | Ku80 | 216780641 | 36 | 128 |
| rs207887 | chr2 | Ku80 | 216783413 | 36 | 128 |
| rs207892 | chr2 | Ku80 | 216786672 | 36 | 128 |
| rs9514825 | chr13 | LIG4 | 107650277 | 36 | 128 |
| rs1105451 (merged | chr13 | LIG4 | 107651324 | 36 | 128 |
| from rs4635191) | | | | | |
| rs868284 | chr13 | LIG4 | 107652214 | 36 | 128 |
| rs9587527 | chr13 | LIG4 | 107653568 | 36 | 128 |
| rs1151402 | chr13 | LIG4 | 107656031 | 36 | 128 |
| rs10131 | chr13 | LIG4 | 107657847 | 36 | 128 |
| rs3093772 | chr13 | LIG4 | 107658205 | 36 | 128 |
| rs1805386 | chr13 | LIG4 | 107659914 | 36 | 128 |
| rs12428162 | chr13 | LIG4 | 107669916 | 36 | 128 |
| rs11069723 | chr13 | LIG4 | 107676482 | 36 | 128 |
| rs3783118 | chr13 | LIG4 | 107682466 | 36 | 128 |
| rs2148429 | chr13 | LIG4 | 107683973 | 36 | 128 |
| rs584531 | chr11 | MRE11 | 93784635 | 36 | 128 |
| rs2508678 | chr11 | MRE11 | 93788997 | 36 | 128 |
| rs540514 (merged | chr11 | MRE11 | 93798653 | 36 | 128 |
| from rs1271079) | | | | | |
| rs7117042 | chr11 | MRE11 | 93810623 | 36 | 128 |
| rs604845 | chr11 | MRE11 | 93822337 | 36 | 128 |
| rs654718 | chr11 | MRE11 | 93829763 | 36 | 128 |
| rs529126 | chr11 | MRE11 | 93833951 | 36 | 128 |
| rs641936 | chr11 | MRE11 | 93836908 | 36 | 128 |
| rs533984 | chr11 | MRE11 | 93838920 | 36 | 128 |
| rs12285522 | chr11 | MRE11 | 93841272 | 36 | 128 |
| rs1270146 | chr11 | MRE11 | 93854954 | 36 | 128 |
| rs659349 | chr11 | MRE11 | 93857304 | 36 | 128 |
| rs540199 | chr11 | MRE11 | 93869313 | 36 | 128 |
| rs2509943 | chr11 | MRE11 | 93870905 | 36 | 128 |
| rs610899 | chr11 | MRE11 | 93874229 | 36 | 128 |
| rs2697677 | chr8 | NBN | 91005544 | 36 | 128 |
| rs4541979 | chr8 | NBN | 91007288 | 36 | 128 |
| rs1881469 | chr8 | NBN | 91010075 | 36 | 128 |
| rs2735889 | chr8 | NBN | 91011243 | 36 | 128 |
| rs2734823 | chr8 | NBN | 91012904 | 36 | 128 |
| rs10464867 | chr8 | NBN | 91014774 | 36 | 128 |
| rs14448 | chr8 | NBN | 91015009 | 36 | 128 |
| rs3087624 (merged | chr8 | NBN | 91015127 | 36 | 128 |
| from rs17348116) | | | | | |
| rs9995 | chr8 | NBN | 91015232 | 36 | 128 |
| rs1063054 | chr8 | NBN | 91015777 | 36 | 128 |
| rs2735383 | chr8 | NBN | 91016445 | 36 | 128 |
| rs1063053 | chr8 | NBN | 91016713 | 36 | 128 |
| rs2735384 | chr8 | NBN | 91017449 | 36 | 128 |
| rs2697679 | chr8 | NBN | 91019056 | 36 | 128 |
| rs6998169 | chr8 | NBN | 91019437 | 36 | 128 |
| rs2735386 | chr8 | NBN | 91020279 | 36 | 128 |
| rs1468078 | chr8 | NBN | 91021106 | 36 | 128 |
| rs6470523 | chr8 | NBN | 91024346 | 36 | 128 |
| rs3736639 | chr8 | NBN | 91024800 | 36 | 128 |
| rs1061302 | chr8 | NBN | 91027598 | 36 | 128 |
| rs2308962 | chr8 | NBN | 91027706 | 36 | 128 |
| rs2280780 | chr8 | NBN | 91030879 | 36 | 128 |
| rs1805812 | chr8 | NBN | 91034229 | 36 | 128 |
| rs709816 | chr8 | NBN | 91036887 | 36 | 128 |
| rs1805786 | chr8 | NBN | 91037038 | 36 | 128 |
| rs7010210 | chr8 | NBN | 91039197 | 36 | 128 |
| rs1805818 | chr8 | NBN | 91040038 | 36 | 128 |
| rs2234744 | chr8 | NBN | 91040111 | 36 | 128 |
| rs16786 | chr8 | NBN | 91040864 | 36 | 128 |
| rs867185 | chr8 | NBN | 91044326 | 36 | 128 |
| rs7006322 | chr8 | NBN | 91048013 | 36 | 128 |
| rs769418 | chr8 | NBN | 91051979 | 36 | 128 |
| rs2293775 | chr8 | NBN | 91054509 | 36 | 128 |
| rs1805833 | chr8 | NBN | 91058414 | 36 | 128 |
| rs1805794 | chr8 | NBN | 91059655 | 36 | 128 |
| rs1805841 | chr8 | NBN | 91061846 | 36 | 128 |
| rs1063045 | chr8 | NBN | 91064195 | 36 | 128 |
| rs1805799 | chr8 | NBN | 91065147 | 36 | 128 |
| rs1805800 | chr8 | NBN | 91066515 | 36 | 128 |
| rs13312840 | chr8 | NBN | 91067085 | 36 | 128 |
| rs13312839 | chr8 | NBN | 91067193 | 36 | 128 |
| rs11989795 | chr8 | NBN | 91067491 | 36 | 128 |
| rs2107465 | chr8 | NBN | 91070200 | 36 | 128 |
| rs1805801 | chr8 | NBN | 91071762 | 36 | 128 |
| rs1805804 | chr8 | NBN | 91074364 | 36 | 128 |
| rs4961165 | chr8 | NBN | 91076707 | 36 | 128 |
| rs2097825 | chr8 | NBN | 91080249 | 36 | 128 |
| rs2072656 | chr8 | NBN | 91082524 | 36 | 128 |
| rs1805855 | chr8 | NBN | 91085932 | 36 | 128 |
| rs739719 | chr5 | RAD50 | 131900764 | 36 | 128 |
| rs739718 | chr5 | RAD50 | 131900972 | 36 | 128 |
| rs2706338 | chr5 | RAD50 | 131923748 | 36 | 128 |
| rs2244012 | chr5 | RAD50 | 131929124 | 36 | 128 |
| rs2299014 | chr5 | RAD50 | 131931298 | 36 | 128 |
| rs2522414 | chr5 | RAD50 | 131939746 | 36 | 128 |
| rs10520114 | chr5 | RAD50 | 131976790 | 36 | 128 |
| rs6596087 | chr5 | RAD50 | 131996508 | 36 | 128 |
| rs6871536 | chr5 | RAD50 | 131997773 | 36 | 128 |
| rs2040705 | chr5 | RAD50 | 132002576 | 36 | 128 |
| rs8073498 | chr17 | TP53 | 7510423 | 36 | 128 |
| rs4968204 | chr17 | TP53 | 7511654 | 36 | 128 |
| rs1614984 | chr17 | TP53 | 7512177 | 36 | 128 |
| rs12951053 | chr17 | TP53 | 7518132 | 36 | 128 |
| rs1625895 | chr17 | TP53 | 7518840 | 36 | 128 |
| rs1042522 | chr17 | TP53 | 7520197 | 36 | 128 |
| rs8079544 | chr17 | TP53 | 7520777 | 36 | 128 |
| rs12602273 | chr17 | TP53 | 7523738 | 36 | 128 |
| rs2287497 | chr17 | TP53 | 7533505 | 36 | 128 |
| rs757049 | chr7 | XRCC2 | 151968110 | 36 | 128 |
| rs10807995 | chr7 | XRCC2 | 151968495 | 36 | 128 |
| rs6964582 | chr7 | XRCC2 | 151982395 | 36 | 128 |
| rs3218491 | chr7 | XRCC2 | 151984681 | 36 | 128 |
| rs3218467 | chr7 | XRCC2 | 151990485 | 36 | 128 |
| rs3218458 | chr7 | XRCC2 | 151991117 | 36 | 128 |
| rs3111465 | chr7 | XRCC2 | 151993408 | 36 | 128 |
| rs3111471 | chr7 | XRCC2 | 151993943 | 36 | 128 |
| rs3218426 | chr7 | XRCC2 | 151995526 | 36 | 128 |
| rs3218416 | chr7 | XRCC2 | 151996794 | 36 | 128 |
| rs3218410 | chr7 | XRCC2 | 151998017 | 36 | 128 |
| rs3218403 | chr7 | XRCC2 | 152000129 | 36 | 128 |
| rs3218400 | chr7 | XRCC2 | 152000622 | 36 | 128 |
| rs6966344 | chr7 | XRCC2 | 152001988 | 36 | 128 |
| rs2283101 | chr7 | XRCC2 | 152003533 | 36 | 128 |
| rs3218373 | chr7 | XRCC2 | 152005096 | 36 | 128 |
| rs6970449 | chr7 | XRCC2 | 152011384 | 36 | 128 |
| rs6464268 | chr7 | XRCC2 | 152012083 | 36 | 128 |
| rs6464269 | chr7 | XRCC2 | 152012188 | 36 | 128 |
| rs10234749 | chr7 | XRCC2 | 152018802 | 36 | 128 |
| rs7796764 | chr7 | XRCC2 | 152022698 | 36 | 128 |
| rs13232006 | chr7 | XRCC2 | 152022832 | 36 | 128 |
| rs861546 | chr14 | XRCC3 | 103225393 | 36 | 128 |
| rs2273175 | chr14 | XRCC3 | 103229894 | 36 | 128 |
| rs861544 | chr14 | XRCC3 | 103232016 | 36 | 128 |
| rs861543 | chr14 | XRCC3 | 103232130 | 36 | 128 |
| rs861542 | chr14 | XRCC3 | 103232476 | 36 | 128 |
| rs3212136 | chr14 | XRCC3 | 103232747 | 36 | 128 |
| rs3212103 | chr14 | XRCC3 | 103236536 | 36 | 128 |
| rs861536 | chr14 | XRCC3 | 103237317 | 36 | 128 |
| rs3212079 | chr14 | XRCC3 | 103240218 | 36 | 128 |
| rs1799795 | chr14 | XRCC3 | 103244578 | 36 | 128 |
| rs861529 | chr14 | XRCC3 | 103249067 | 36 | 128 |
| rs861528 | chr14 | XRCC3 | 103252751 | 36 | 128 |
| rs2144078 | chr14 | XRCC3 | 103254100 | 36 | 128 |
| rs10138768 | chr14 | XRCC3 | 103260280 | 36 | 128 |
| rs2295151 | chr14 | XRCC3 | 103262852 | 36 | 128 |
| rs2295148 | chr14 | XRCC3 | 103265363 | 36 | 128 |
| rs2295146 | chr14 | XRCC3 | 103269109 | 36 | 128 |
| rs8548 | chr14 | XRCC3 | 103269333 | 36 | 128 |
| rs3825550 | chr14 | XRCC3 | 103271302 | 36 | 128 |
| rs10514246 | chr5 | XRCC4 | 82401554 | 36 | 128 |
| rs2075685 | chr5 | XRCC4 | 82408421 | 36 | 128 |
| rs2075686 | chr5 | XRCC4 | 82408502 | 36 | 128 |
| rs10462397 | chr5 | XRCC4 | 82421248 | 36 | 128 |
| rs2386235 | chr5 | XRCC4 | 82433027 | 36 | 128 |
| rs1478483 | chr5 | XRCC4 | 82437062 | 36 | 128 |
| rs1120476 | chr5 | XRCC4 | 82458399 | 36 | 128 |
| rs6860239 | chr5 | XRCC4 | 82467597 | 36 | 128 |
| rs1382367 | chr5 | XRCC4 | 82486968 | 36 | 128 |
| rs2061783 | chr5 | XRCC4 | 82491914 | 36 | 128 |
| rs2662241 | chr5 | XRCC4 | 82520349 | 36 | 128 |
| rs3777041 | chr5 | XRCC4 | 82536051 | 36 | 128 |
| rs3734091 | chr5 | XRCC4 | 82536490 | 36 | 128 |
| rs10514249 | chr5 | XRCC4 | 82540612 | 36 | 128 |
| rs7711825 | chr5 | XRCC4 | 82557374 | 36 | 128 |
| rs963248 | chr5 | XRCC4 | 82569650 | 36 | 128 |
| rs1193695 | chr5 | XRCC4 | 82578842 | 36 | 128 |
| rs301276 | chr5 | XRCC4 | 82583487 | 36 | 128 |
| rs301275 | chr5 | XRCC4 | 82598817 | 36 | 128 |
| rs3885676 | chr5 | XRCC4 | 82609001 | 36 | 128 |
| rs3777036 | chr5 | XRCC4 | 82611352 | 36 | 128 |
| rs3777033 | chr5 | XRCC4 | 82615193 | 36 | 128 |
| rs40123 | chr5 | XRCC4 | 82622245 | 36 | 128 |
| rs3777028 | chr5 | XRCC4 | 82625086 | 36 | 128 |
| rs10514253 | chr5 | XRCC4 | 82625806 | 36 | 128 |
| rs301282 | chr5 | XRCC4 | 82636361 | 36 | 128 |
| rs301286 | chr5 | XRCC4 | 82638711 | 36 | 128 |
| rs445403 | chr5 | XRCC4 | 82642878 | 36 | 129 |
| rs7728486 | chr5 | XRCC4 | 82666339 | 36 | 128 |
| rs10061326 | chr5 | XRCC4 | 82673080 | 36 | 128 |
| rs10434637 | chr5 | XRCC4 | 82675426 | 36 | 128 |
| rs7735781 | chr5 | XRCC4 | 82676233 | 36 | 128 |
| rs10057194 | chr5 | XRCC4 | 82694327 | 36 | 128 |

What is claimed is:

1. A method of predicting the health of a subject, the method comprising:
obtaining nucleic acid sequence data about the subject;
identifying at least one polymorphic risk marker associated with a change in promoter methylation of a gene associated with lung cancer; and
predicting the health of the subject from a presence of at least one polymorphic risk marker identified.

2. The method of claim 1 wherein obtaining a nucleic acid sequence data is obtained for one or more of the flowing genes XRCC3, DNA-PKc, NBN, LIG4, XRCC2, CHEK1, MRE11A, CHEK2, RAD50, and KU80.

3. The method of claim 1 wherein a gene associated with cancer is selected from the group consisting of p16, MGMT, DAPK, RASSF1A, PAX5-α, PAX5-β, GATA4, and GATA5.

4. The method of claim 1 wherein the at least one polymorphic risk marker is selected from the group consisting of: an allele A in marker rs537046 of gene CHEK1; an allele C in marker rs5762763 of gene CHEK2; an allele C in marker rs1151402 of gene LIG4; an allele Tin marker rs7117042 of gene MRE11A; an allele Tin marker rs6998169 of gene NBN; an allele A in marker rs7830743 of gene DNA-PKc; an allele G in marker rs2244012 of gene RAD50; an allele C in marker rs3218400 of gene XRCC2; an allele C in marker rs2295146 of gene XRCC3; and an allele A in marker rs828911 of gene KU80.

5. The method of claim 1, wherein determining the health of a subject comprises comparing the obtained nucleic acid sequence data to a database containing correlation data between polymorphic risk markers and risk factors to provide a score relating to the health of the subject.

6. The method of claim 4 wherein determining a risk includes identifying the presence of a five polymorphic risk markers selected from the group consisting of: an allele C in marker rs5762763 of gene CHEK2; an allele T in marker rs7117042 of gene MRE11A; an allele T in marker rs6998169 of gene NBN; an allele A in marker rs7830743 of gene DNA-PKc; and an allele C in marker rs2295146 of gene XRCC3.

7. The method of claim 6 wherein the presence of the five polymorphic risk markers from the group are present in 7 or more of 10 possible alleles.

8. The method of claim 4 further comprising detecting a polymorphic risk marker that is in linkage disequilibrium with one or more of the at least one polymorphic risk markers identified in claim 4.

9. The method of claim 8 wherein the polymorphic risk markers in linkage disequilibrium with a polymorphic risk marker are selected from table 7.

10. The method of claim 8 wherein linkage disequilibrium is defined by numerical values of r.̂2 of at least 0.8.

11. The method of claim 1 further comprising detecting in a nucleic acid sample of the subject a polymorphic risk marker for one or more of the genes selected from the group consisting of XRCC3, DNA-PKc, NBN, LIG4, XRCC2, CHEK1, MRE11A, CHEK2, RAD50, and KU80.

12. A kit for detecting a polymorphic risk marker associated with a change in promoter methylation of a gene comprising:
reagents for selectively detecting at least one allele of at least one polymorphic risk marker from XRCC3, DNA-PKc, NBN, LIG4, XRCC2, CHEK1, MRE11A, CHEK2, RAD50, and KU80 in the genome of an individual, wherein the polymorphic risk marker is selected from the group consisting of the polymorphic risk markers listed in Table 7, and markers in linkage disequilibrium therewith.

13. A computer-readable medium having computer executable instructions for predicting the health of a subject at risk for developing lung cancer the computer readable medium comprising:
data indicative of at least one polymorphic risk marker from each gene selected from the group consisting of XRCC3, DNA-PKc, NBN, LIG4, XRCC2, CHEK1, MRE11A, CHEK2, RAD50, and KU80;
a routine stored on the computer readable medium and adapted to be executed by a processor to predict the health of a subject at risk for developing lung cancer when one or more from the at least one polymorphic risk marker from at least one gene selected from the group consisting of XRCC3, DNA-PKc, NBN, LIG4, XRCC2, CHEK1, MRE11A, CHEK2, RAD50, and KU80 is present in a nucleic acid sequence data obtained from a subject.

14. The routine on the computer readable medium of claim 13 further comprising identifying the presence of five polymorphic risk markers selected from the group consisting of an allele C in marker rs5762763 of gene CHEK2; an allele Tin marker rs7117042 of gene MRE11A; an allele Tin marker rs6998169 of gene NBN; an allele A in marker rs7830743 of gene DNA-PKc; and an allele C in marker rs2295146 of gene XRCC3.

15. The routine on the computer readable medium of claim 13b wherein identifying the presence of the five polymorphic risk markers includes identifying the five polymorphic risk markers in 7 or more of 10 possible alleles.

16. The routine on the computer readable medium of claim 13 further comprising detecting from the nucleic acid sequence data a polymorphic risk marker that is in linkage disequilibrium with one or more of the at least one polymorphic risk markers identified in claim 13

17. A method of aiding in a diagnosis of a subject suspected of lung cancer, the method comprising the steps of:
obtaining nucleic acid sequence data about the subject;
identifying the presence of one or more polymorphic risk markers from the nucleic acid sequence data;
comparing the number of polymorphic risk markers to a look up table and assigning a score based upon the number of polymorphic risk markers present;
determining whether said subject has a risk of lung cancer based on the score.

18. The method of claim 17, wherein the one or more polymorphic risk markers are selected from the group consisting of an allele A in marker rs537046 of gene CHEK1; an allele C in marker rs5762763 of gene CHEK2; an allele C in marker rs1151402 of gene LIG4; an allele Tin marker rs7117042 of gene MRE11A; an allele Tin marker rs6998169 of gene NBN; an allele A in marker rs7830743 of gene DNA-PKc; an allele G in marker rs2244012 of gene RAD50; an allele C in marker rs3218400 of gene XRCC2; an allele C in marker rs2295146 of gene XRCC3; and an allele A in marker rs828911 of gene KU80.

19. The method of claim 17, further comprising obtaining at least one biometric parameter from the subject.

20. The method of claim 19, wherein the at least one biometric parameter is based on the smoking history of the subject.
